(19) United States
(12) Patent Application Publication
(10) Pub. No.: US 2025/0269031 A1
(43) Pub. Date: Aug. 28, 2025

(54) MITOCHONDRIA TARGETING QUINOLINIUM-DRUG CONJUGATES AND THEIR SELF-ASSEMBLING NANOFORMULATIONS FOR CANCER THERAPY

(71) Applicant: THE REGENTS OF THE UNIVERSITY OF CALIFORNIA, Oakland, CA (US)

(72) Inventors: Yuanpei LI, Oakland, CA (US); Zhao MA, Oakland, CA (US)

(21) Appl. No.: 18/254,292

(22) PCT Filed: Nov. 23, 2021
(86) PCT No.: PCT/US2021/060548

§ 371 Date: May 24, 2023

Related U.S. Application Data
(60) Provisional application No. 63/117,638, filed on Nov. 24, 2020.

(51) Int. Cl. A61K 41/00; A61K 47/54

(52) U.S. Cl. CPC A61K 41/0076; A61K 47/545

(57) ABSTRACT

The present invention proves mitochondria-targeting nanofibers that are formed by assembly of small-molecule building blocks of amphiphilicity to improve photodynamic cancer therapy. Monomers derived from a pheophorbide a and quinolinium conjugates are described, as well as the formation of nanoparticles, formulations of the compounds, and methods of treatment.

### CROSS-REFERENCES TO RELATED APPLICATIONS

This application claims priority to U.S. Provisional Application No. 63/117,638, filed Nov. 24, 2020, which is incorporated herein in its entirety for all purposes.

### BACKGROUND OF THE INVENTION

Photodynamic therapy (PDT) has emerged as an attractive alternative in cancer therapy, but its therapeutic effects are limited by the nonselective subcellular localization and poor intratumoral retention of small-molecule photosensitizes. Here a fiber-forming nanophotosensitizer (PQC NF) that is composed of mitochondria targeting small molecules of amphiphilicity is reported. Harnessing the specific mitochondria targeting, the light-activated PQC NFs produce approximately 110-fold higher amount of reactive oxygen species (ROS) in cells than free photosensitizers and can dramatically induce mitochondrial disruption to trigger intense apoptosis, showing 20-50 times better in vitro anticancer potency than traditional photosensitizers. As fiber-shaped nanomaterials, PQC NFs also demonstrated a long-term retention in tumor sites, solving the challenge of rapid clearance of small-molecule photosensitizers from tumors. With these advantages, PQC NFs achieve a 100% complete cure rate in both subcutaneous and orthotopic oral cancer models with the administration of only a single dose. This type of single small molecule-assembled mitochondria targeting nanofibers offer an advantageous strategy to improve the in vivo therapeutic effects of conventional PDT.

Photodynamic therapy (PDT) is a well-established clinical treatment modality for cancer, which combines the photosensitizer, light energy, and oxygen to produce singlet oxygen (¹O₂) and trigger a chain of reactions of reactive oxygen species (ROS) leading to cell death. The technique is gaining popularity due to its minimally invasive nature for patients, short-course treatment, and selective cytotoxicity. Since ¹O₂ has a short lifetime (˜3 μs) and a limited diffusion radius (0.02 μm), the photosensitizer only causes photodamage in its direct vicinity. Therefore, the efficiency of PDT is strongly dependent on the intracellular accumulation and subcellular localization of photosensitizers. To address this challenge, a promising solution is delivering the photosensitizers to specific organelles, where ¹O₂ is generated in situ to efficiently trigger phototoxicity. As the main powerhouse and important ROS source in cells, the mitochondrion is a potentially excellent target for PDT. Since the inner mitochondrial membrane has a strong negative membrane potential, mitochondria targeting PDT can be achieved by conjugating photosensitizers to delocalized cations, such as triphenylphosphonium (TPP) and dequalinium (DQA). Several reported mitochondria-targeted photosensitizers have shown significantly improved therapeutic effects relative to their free photosensitizer.

Another limitation for traditional PDT is the rapid clearance of the small-molecule photosensitizers from tumors, which causes the poor retention of photosensitizers in tumor cells and insufficient therapeutic effects in vivo. Self-assembling nanomaterials are retained longer at local sites, which gives the potential to overcome this limitation of photosensitizer clearance. Through supramolecular self-assembly, small-molecule drugs can be functionalized with superior nanoscale characteristics and self-delivering properties. This carrier-free nanomedicine strategy urges the development of small-molecule nanophotosensitizers. However, most of the developed nanophotosensitizers are spherical nanoparticles, not nanofibers. Compared to the traditional spherical nanoparticles, the fiber-forming nanomedicines are revolutionizing the field of drug delivery due to their high surface-area-to-volume ratio, small inter-fibrous pore size with high porosity, and enhanced retention effects. Among the attempts to construct nanofibers, the majority of building blocks are β-sheet peptide-based motifs that are utilized to drive supramolecular assembly and hydrogel formation. Although these peptide assemblies have demonstrated great advantages of nanofibers in medical diagnosis and therapy, there remain practical and system-specific challenges in the manufacture of peptide materials and complex self-assembly process. Since these limitations can be overcome by small-molecule drugs, recently small molecule-based nanofibers have drawn attention. However, due to the lack of available fiber-forming small-molecule monomers, there are only limited reported cases to date.

Herein, according to the above strategies to improve PDT and principles of new drug design and molecular self-assembly, we developed a mitochondria-targeting nanofiber (PQC NF) that are formed by self-assembly of small-molecule building blocks of amphiphilicity (FIG. 1) for the photodynamic cancer therapy. The monomer is a pheophorbide a (PA) and quinolinium conjugate (PQC), in which the hydrophobic PA acts as the photosensitization group and the quinolinium moiety is hydrophilic cation for mitochondria targeting. PQC NFs could specifically accumulate in mitochondria and are retained there for an extended period, where they exhibited a powerful PDT effect to induce mitochondrial disruption and lead to apoptotic cell death. When compared with free PA, PQC NFs showed a 20-50-fold increase in cytotoxicity in vitro and can be retained within tumor sites in vivo for 10 days. Using these advantages, PQC NFs achieved a powerful tumor ablation effect in both subcutaneous and orthotopic oral cancer models when treated with only a single dose. As fiber-shaped nanophotosensitizers that are self-assembled from mitochondria-targeted small molecules, PQC NFs provide a useful strategy to chemically modify existing photosensitizers to enhance their phototherapeutic effects.

An amphiphilic photosensitizer-derived small molecule that can self-assemble into a fiber-forming nanoconstruct was developed. The one-component nanofiber not only exhibits a significantly phototoxicity to cancer cells through targeting mitochondria, but also shows the long-term retention in tumor site, thus achieving a 100% complete cure rate in both subcutaneous and orthotopic oral cancer models with only a single-dose administration.

Mitochondria are implicated in multiple aspects of tumorigenesis, tumor progression, and tumor resistance. In order to adapt to the internal and external environment, tumor cells change their mitochondria structurally and functionally, which are different from the normal counterparts. Tumor cells also exhibit an extensive metabolic reprogramming that renders them more susceptible to mitochondrial perturbations than non-immortalized cells. Therefore, targeting mitochondrial bioenergetics is emerging as a viable approach to inhibit the growth of cancer cells. There are a lot of existing chemotherapeutic and phototherapeutic compounds that can inhibit cancer cells by interacting with mitochondrial target or destroying mitochondria. However, those compounds have a poor capacity to target mitochondria, which limits their therapeutic outcome. Linking the chemotherapy drug or photosensitizers to a mitochondrial targeting moiety is a promising strategy to improve activity and toxicity profiles. Furthermore, owing to hydrophobicity of the aforementioned drugs, it is not easy to make a stable and convenient formulation. Balancing the hydrophilic and hydrophobic properties of the parental drugs to form nanostructures can optimize drug formulation and improve drug systemic delivery toward the tumor site.

To address these limitations, herein, we used the hydrophilic quinolinium group with mitochondria targeting property to conjugate the hydrophobic drugs that can prefer to function in mitochondria, thus obtaining a list of amphiphilic quinolinium-drug conjugates. These conjugates can self-assemble into interesting nanostructures, such as nanofiber or micelles, with positive surface charges. Compared the small-molecule parental drugs, the assembled nanodrugs show enormous advantages of improvement of physical formulations and anticancer activities. For these nano-assemblies, we still have some space to improve their properties. Hence, we then modify the quinolinium group by using the maleic acid-derived group, a tumor acidity-responsive moiety to design a prodrug form for the quinolinium-drug conjugates. The prodrug nanoparticles have negative surface charges and can achieve longer blood circulation. Upon arriving at the tumor site, the prodrug nanoparticles will lose maleic acid moieties and increase zeta potential by responding to tumor acidity, which significantly enhances cellular uptake and improves the in vivo tumor inhibition.

### BRIEF SUMMARY OF THE INVENTION

In one embodiment, the present invention provides a compound of Formula (I):

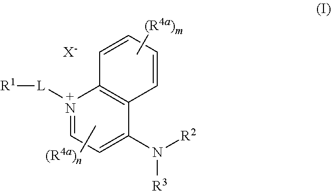

wherein: R¹ is a hydrophobic drug or photosensitizer; L is C₂₋₂₀ alkylene, C₂₋₂₀ alkenylene, C₂₋₂₀ alkynylene, or polyethyleneglycol polymer; R² is H, C₁₋₆ alkyl,

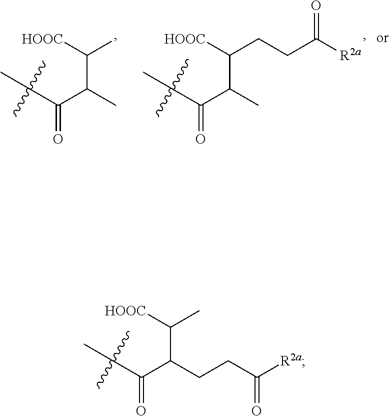

R^{2a} is —OH, —NH-polyethyleneglycol, polyethyleneglycol or a peptide; R³ is H or C₁₋₆ alkyl; each R^{4a} and R^{4b} is independently H, C₁₋₆ alkyl, C₂₋₆ alkenyl, C₂₋₆ alkynyl, halogen, or C₁₋₆ haloalkyl; subscript m is from 1 to 4; subscript n is from 1 to 2; and X is Cl, Br or I; wherein when R¹ is cyclosporin, R² is Me, and R³ is Me, then L is C₂₋₂₀ alkylene, C₁₀₋₂₀ alkenylene, C₂₋₂₀ alkynylene, or polyethyleneglycol polymer.

In another embodiment, the present invention provides a nanofiber comprising a plurality of conjugates of Formula (I):

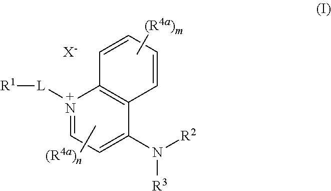

wherein: R¹ is a hydrophobic drug or photosensitizer; L is C₂₋₂₀ alkylene, C₂₋₂₀ alkenylene, C₂₋₂₀ alkynylene, or polyethyleneglycol polymer; R² is H, C₁₋₆ alkyl,

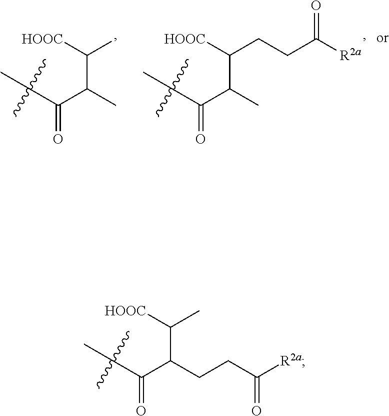

R^{2a} is —OH, —NH-polyethyleneglycol, polyethyleneglycol or a peptide; R³ is H or C₁₋₆ alkyl; each R^{4a} and R^{4b} is independently H, C₁₋₆ alkyl, C₂₋₆ alkenyl, C₂₋₆ alkynyl, halogen, or C₁₋₆ haloalkyl; subscript m is from 1 to 4; subscript n is from 1 to 2; and X is Cl, Br or I.

In another embodiment, the present invention provides a nanoparticle comprising a plurality of conjugates of Formula (I):

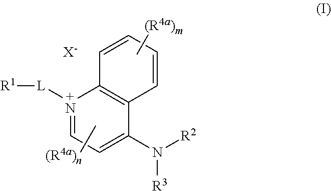

wherein: R¹ is a hydrophobic drug or photosensitizer; L is C₂₋₂₀ alkylene, C₂₋₂₀ alkenylene, C₂₋₂₀ alkynylene, or polyethyleneglycol polymer; R² is H, C₁₋₆ alkyl,

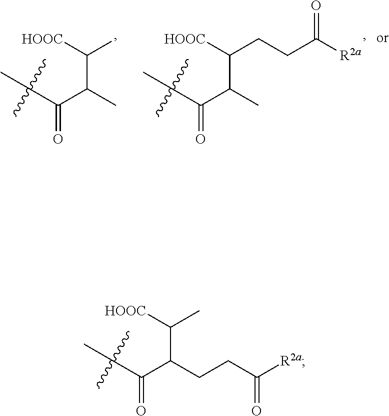

R^{2a} is —OH, —NH-polyethyleneglycol, polyethyleneglycol or a peptide; R³ is H or C₁₋₆ alkyl; each R^{4a} and R^{4b} is independently H, C₁₋₆ alkyl, C₂₋₆ alkenyl, C₂₋₆ alkynyl, halogen, or C₁₋₆ haloalkyl; subscript m is from 1 to 4; subscript n is from 1 to 2; and X is Cl, Br or I.

In another embodiment, the present invention provides a method of treating a disease, the method comprising administering a therapeutically effective amount of a nanofiber comprising a plurality of conjugates of Formula (I) or a nanoparticle comprising a plurality of conjugates of Formula (I) to a subject in need thereof.

In another embodiment, the present invention provides a method of treating a disease via photodynamic therapy, the method comprising administering a therapeutically effective amount of a nanofiber comprising a plurality of conjugates of Formula (I) or a nanoparticle comprising a plurality of conjugates of Formula (I), wherein R¹ is a photosensitizer, to a subject in need thereof.

### BRIEF DESCRIPTION OF THE DRAWINGS

FIG. 1 Schematic illustration of single small molecule-assembled mitochondria targeting nanofibers (PQC NFs). The PQC monomer is a conjugate of pheophorbide a (PA) and quinolinium. PQC NFs exhibited nanomolar cytotoxicity by mediating mitochondria-targeting phototherapy and were retained long-term at the tumor site. With these advantages, PQC NFs achieved robust anticancer effects in vivo with a 100% complete cure rate after the administration of only a single dose.

FIG. 2A-FIG. 2H shows (FIG. 2A) Chemical structures of DQA, PA and PQC. (FIG. 2B) Absorbance spectra of DQA (5 μM), PA (10 μM), and PQC (10 μM) in methanol. (FIG. 2C) Fluorescent spectra of PA and PQC (10 μM) in methanol (λex=412 nm). (FIG. 2D) Transmission electron microscope (TEM) photograph of PQC NFs. Scale bar=200 nm. (FIG. 2E) Appearance and fluorescence spectra of PQC NFs after centrifugal filtration (10 kDa). The working concentration is 2 mM for centrifugation, and the spectra were measured after dilution with methanol (1:500). (FIG. 2F) Fluorescence spectra of PQC NFs or PA (20 μM) in the assembly (PBS) and dissociation (PBS/SDS) forms. (FIG. 2G) Fluorescence imaging under Cy5 channel of PQC NFs or PA in the assembly (PBS) and dissociation (PBS/SDS) forms. (FIG. 2H) Singlet oxygen production of PA and PQC NFs measured by using SOSG as an indicator. The solutions of PA and PQC NFs in PBS and PBS/SDS were exposed to the NIR light (30 mW cm⁻²) for 60 s.

FIG. 3A-FIG. 3H shows (FIG. 3A) Cell viability. OSC-3 cells were incubated as indicated for 24 h and then were treated with or without light treatment (30 mW cm² for 30 s), followed by another 24 h incubation. (FIG. 3B) Time-course of cellular uptake for PA and PQC NFs (1 μM) in OSC-3 cells. (FIG. 3C, FIG. 3D) Influence of temperature (FIG. 3C) and various inhibitors (FIG. 3D) on the endocytosis of PQC NFs. (FIG. 3E) Representative fluorescence images of the time-dependent localization for PQC NFs (2 μM) in OSC-3 cells. Scale bar=10 μm. Images were captured under the condition of the best signal for the accurate colocalization analysis, therefore, the brightness did not represent the relative fluorescence intensity of individual images. (FIG. 3F) Calculated Pearson correlation coefficient (Pearson's R) for colocalization analysis of images in (FIG. 3E). (FIG. 3G) Cellular distribution of free PA. OSC-3 cells were incubated with PA (2 μM) for 4 h. Scale bar=10 μm. (FIG. 3H) Fluorescence ratio of PA or PQC in mitochondria and cytoplasm. Mitochondria and cytoplasm fractions were isolated from OSC-3 cells that were pretreated with PA and PQC NFs (1 μM) for 24 h.

FIG. 4A-FIG. 4F shows (FIG. 4A) Flow cytometry analysis of ROS levels in OSC-3 cells using DCF-DA as an indicator. (FIG. 4B) Mitochondrial membrane potential analysis of OSC-3 cells that were treated as indicated (1 μM) and stained with JC-1. (FIG. 4C) Quantitative red to green fluorescence ratio of cells in (FIG. 4B). (FIG. 4D) Representative TEM graphs showing morphological changes of mitochondria in OSC-3 cells that were treated as indicated (0.5 μM, 24 h). The green and red arrows designate the normal and damaged mitochondria, respectively. Scale bars are 5 μm (upper panel) and 200 nm (lower panel). (FIG. 4E) Apoptosis assay of OSC-3 cells within the indicated treatments (0.2 μM). (FIG. 4F) Changes of apoptosis-related proteins, including cytochrome C, PARP, and caspase 3, in OSC-3 cells that were treated with PQC NFs (0.5 μM) with or without light. The above-mentioned light treatment was performed for 30 s using a 633-nm LED array at a power density of 30 mW cm⁻² at room temperature.

FIG. 5A-FIG. 5l shows (FIG. 5A) Time-course in vivo fluorescence imaging of mice bearing the subcutaneous OSC-3 tumor. Mice were treated with PA or PQC NFs via intratumoral injection (10 nmol per 50 mm³) and were observed at the indicated time points. (FIG. 5B) Intratumoral ROS levels that were measured by ex vivo imaging at different intervals post-injection of PA or PQC NFs, in which DCF-DA was used as an indicator. (FIG. 5C) The establishment of subcutaneous (up) and orthotopic (down) oral tumor models, and the treatment schedules. Drugs (10 nmol per 50 mm³) were injected intratumorally at Day 0. The subcutaneous tumors in right flank and the orthotopic tumors were then treated with laser on Day 1, Day 2, Day 5, and Day 6. The laser (680 nm) doses were all set as 0.2 W cm⁻² for 6 min. (FIG. 5D) Tumor growth curves for the subcutaneous tumor model (n=6). (FIG. 5E) Tumor growth curves for the orthotopic tumor model (n=6). The relative tumor volume is the ratio of the absolute volume of the respective tumor on day x to the absolute volume of the same tumor on day 0. (FIG. 5F) Body weight changes of mice (n=6) for the orthotopic tumor model. (FIG. 5G) Representative TEM graphs of subcutaneous tumors that were treated with PA or PQC NFs for 24 h, followed with laser treatment. The green and red arrows designate the normal and damaged mitochondria, respectively. (FIG. 5H, FIG. 5I) Representative results of H&E (FIG. 5H) and Ki67-IHC. (FIG. 5I) Staining analysis of subcutaneous tumors that were treated with PA or PQC NFs for 24 h, followed by laser treatment or not. Scale bar=100 μm.

FIG. 6 shows the synthetic route of PQC monomer.

FIG. 7 illustrates the critical aggregation concentration (CAC) of PQC NFs was determined using dynamic light scattering (DLS).

FIG. 8 shows MALDI-TOF mass spectrometric analysis of PQC (top) and DQA (bottom). The MALDI-TOF mass spectra of PQC showing the monomers (m/z 888.517), dimers (m/z 1777.034), tetramers (m/z/4 1184.689) and heptamers (m/z/6 1036.603). The MALDI-TOF mass spectra of DQA showing the monomers (m/z 456.324).

FIG. 9 shows characterization of PA aggregate in PBS that was measured by using DLS.

FIG. 10 shows viability results of OSC-3 cells that were treated with different nanoformulations of PA derivatives for 24 h, followed by light treatment (30 mW cm⁻² for 30 s) and another 24 h incubation.

FIG. 11A-FIG. 11B shows viability results of pancreatic cancer cells (BXPC-3, AsPC-1, and PANC-1), bladder cancer cells (UM-UC-3 and 5637), and noncancerous cells (IMR90) with the indicated treatments. (FIG. 11A) Cell viability curves, (FIG. 11B) IC₅₀ values of cytotoxicity.

FIG. 12 shows time-dependent monitoring of cellular uptake and distribution of PQC NFs in OSC-3 cells. Cell were incubated with PQC NFs (1 μM). Scale bar=20 μm.

FIG. 13A-FIG. 13B shows cellular uptake of PQC NFs (1 μM) that was intervened by (FIG. 13A) low temperature (4° C.) and (FIG. 13b) endocytosis inhibitors: sodium azide (1 mg mL⁻¹), chlorpromazine (20 μg mL⁻¹), genistein (10 μg mL⁻¹) and amiloride (50 μM). Scale bar=100 m.

FIG. 14 shows the TEM observation of the assembled and dissociated of PQC NFs (100 μM) that were incubated with the freshly isolated mitochondria (0.1 mg mL⁻¹), lysosomes (0.1 mg mL⁻¹), and other cellular components (0.1 mg mL⁻¹) for 24 h, respectively.

FIG. 15 shows mitochondrial membrane potential analysis of OSC-3 cells by JC-1 staining. Cells were treated with Pa (4 μM) for 24 h and, exposed to light (30 mW cm⁻²) for 30 s, followed by further incubation for 2 h. Scale bar=20 μm.

FIG. 16 shows apoptosis assay of OSC-3 cells that were treated with 2 μM of Pa or PQC NFs for 24 h, followed by light treatment (30 mW cm⁻², 30 s) and another incubation for 12 h.

FIG. 17A-FIG. 17C shows absorption spectra (FIG. 17A), fluorescence spectra (FIG. 17B), and fluorescence imaging under different channels (FIG. 17C) of 5 μM of DCF-DA, DCF, PA, and PQC NFs in PBS with 5% SDS. DCF represents the activated DCF-DA, which is prepared by incubating the DCF-DA solution (5 μM) with hydrogen peroxide (10 μM) and lipase (0.1 mg mL⁻¹) for 30 min.

FIG. 18 shows ex vivo fluorescence imaging of ROS in OSC-3 tumors. For Pre group, mice were irradiated with laser (0.2 W cm⁻², 6 min) at tumor sites. For other treatment groups, mice were intratumorally injected with Pa or PQC NFs (10 nmol per 50 mm³ tumor), and irradiated with laser at Days 1, 2, 5, 6 post injection. Tumors were collected immediately post laser treatment and were cut into small pieces with a similar volume of 60 mm³, which were further stained with DCF-DA for fluorescence imaging and quantification.

FIG. 19A-FIG. 19B shows (FIG. 19A) Tumor temperature images captured by FUR thermal camera. Mice were treated intratumorally as indicated (10 nmol per 50 mm³ tumor), and tumors were irradiated with laser (0.2 W cm⁻², 6 min) at 24 h post injection, followed by temperature measurement. (FIG. 19B) Temperature changes (ΔT) of tumors for each group.

FIG. 20 shows H&E staining of main organs illustrated the systemic toxicity of each treatment in comparison with the vehicle group. Scale bar=100 μm.

FIG. 21A-FIG. 21J shows (FIG. 21A) Chemical structures of PA and PQC. (FIG. 21B) Schematic synthesis of LPHNPs. (FIG. 21C, FIG. 21D) Representative TEM images of PQC NFs (FIG. 21C) and LPHNPs (FIG. 21D), scale bar=50 nm. (FIG. 21E) DLS analysis of LPHNPs. (FIG. 21F) Stability measurements of LPHNPs in 7 days. (FIG. 21G) Representative TEM image of liposome@PA, Scale bar=50 nm. (FIG. 21H) DLS of liposome@PA, scale bar=50 nm. (FIG. 21l) Stability measurements of liposome@PA in 7 days. (FIG. 21J) ¹O₂ production levels of different formulations of PQC or PA in the aggregation (PBS) and dissociation (PBS/SDS) forms.

FIG. 22A-FIG. 22l shows (FIG. 22A) Time-dependent uptake for liposome@PA and LPHNPs (1 μM) in GL261 cells. (FIG. 22B) Viability curves of GL261 cells treated with PA, PQC NFs, liposome@PA and LPHNPs, with or without light irradiation. (FIG. 22C) Fluorescence colocalization of LPHNPs (1 μM) and mitochondria in GL261 cells, scale bar=20 μm. (FIG. 22D) Pearson correlation coefficient for colocalization analysis and the mean fluorescence intensity in cells. (FIG. 22E) Fluorescent imaging of ROS in GL261 cells treated as indicated (0.5 μM), scale bar=50 μm. (FIG. 22F) Fluorescence quantitative analysis of ROS in FIG. 22E. (FIG. 22G) JC-1 imaging to analyze the mitochondrial membrane potential of cells that were treated as indicated, scale bar=20 μm. (FIG. 22H) the corresponding quantitative analysis of red to green fluorescence intensity ratio of cells in FIG. 22G. (FIG. 22l) Representative TEM images of GL261 cells that were treated as indicated (0.5 μM, 24 h), scale bar: 2 μm (upper) and 0.2 μm (lower), arrows: mitochondria.

FIG. 23A-FIG. 23D shows (FIG. 23A) Bioluminescence imaging of orthotopic GL261 tumors and fluorescence biodistribution of LPHNPs (10 mg/kg) in living mice at different time point after injection. (FIG. 23B) Ex vivo fluorescence imaging to show biodistribution of LPHNPs among tumor and major organs. (FIG. 23C) Confocal images of cryosection of harvested brain tissues from the orthotopic GL261 mouse model at 48 h post-injection with LPHNPs. Green: GL261-GFP, blue: Hoechst 33342, red: LPHNPs, scale bar=1 mm. (FIG. 23D) Multichannel fluorescence imaging of local brain bearing the orthotopic GL261 tumor. Green: GL261-GFP, red: LPHNPs, scale bar=4 mm.

FIG. 24A-FIG. 24F shows (FIG. 24A) Establishment of orthotopic GL261 model and treatment schedule with PBS, liposome@PA (10 mg/kg) with laser, LPHNPs (10 mg/kg) with laser. Laser dose was set as 0.2 W/cm² for 3 min. (FIG. 24B) Quantitative data from bioluminescence imaging (FIG. 24C) representative bioluminescence images of GL261-bearing mice treated with PBS, liposome@PA with laser and LPHNPs with laser, n=5. (FIG. 24D) Kaplan-Meier survival curve of the GL261-bearing mice receiving the indicated treatments, n=5. (FIG. 24E) H&E staining of the harvested brain tissues, in which the heavily stained refers to the glioma area, Scale bar=20 μm. (FIG. 24F) Calculated areas of GL261 tumors from three treatment groups. **p<0.01, *p<0.05.

FIG. 25A-FIG. 25E shows (FIG. 25A) Zeta potential of PQC NFs, liposome@PA and LPHNPs. (FIG. 25B) Molecular weight cut-off and UV-Vis absorbance of LPHNPs (MWCO: 10 kDa). (FIG. 25C, FIG. 25D) DLS analysis of LPHNPs (FIG. 25C) and liposome@PA (FIG. 25D) in the presence of 10% FBS. (FIG. 25E) Appearance of LPHNPs (loading rate: 20%) and liposome@PA (loading rate: 20%) after placed for 7 days, arrow: precipitate of liposome@PA.

FIG. 26A-FIG. 26C shows (FIG. 26A, FIG. 26B UV-Vis (FIG. 26A) and fluorescence (FIG. 26B) spectra of LPHNPs or liposome@PA (20 PM) in the aggregation (PBS) and dissociation (PBS/SDS) forms. (FIG. 26C) Fluorescence imaging (Cy5 channel) of LPHNPs, liposome@PA, PQC and PA in the aggregation (PBS) and dissociation (PBS/SDS) forms.

FIG. 27A-FIG. 27C shows viabilities of U251 (FIG. 27A) and U118 (FIG. 27B) cells that were treated as indicated. (FIG. 27C) IC₅₀ values.

FIG. 28A-FIG. 28B shows (FIG. 28A) Confocal images of mito-ROS in cells treated as indicated. scale bar=50 m. (FIG. 28b) Quantitative red fluorescence intensity of cells in (FIG. 28A).

FIG. 29A-FIG. 29C shows (FIG. 29A) Body weight changes of mice that were i.v. injected with PBS or LPHNPs (10 mg/kg) at on first and third days. (FIG. 29B, FIG. 29C) H&E staining of main organs (FIG. 29B) and hematology analysis (FIG. 29C) of mice that were treated with 10 mg/kg LPHNP. scale bar=100 μm

FIG. 30 shows H&E staining of main organs from orthotopic GL261 model treated with PBS, 10 mg/kg liposome@PA NPs and laser, 10 mg/kg LPHNPs and laser, scale bar=100 μm.

FIG. 31A-FIG. 31D shows (FIG. 31A) Chemical structures of LM, LND and m104. (FIG. 31B) Viability curves of pancreatic cancer stem cell (CSC) treated as indicated. (FIG. 31C) Cell growth of CSC treated with LM, LND and m104 (2 μM). (FIG. 31D) Clonogenic assay of CSC cells treated as indicated.

FIG. 32A-FIG. 32E shows viability curves of pancreatic cancer cells (FIG. 32A) Bxcp-3, (FIG. 32B) AsPc, (FIG. 32C) Paca-2 and (FIG. 32D) PANC-1 treated as indicated. (FIG. 32E) IC₅₀ values of cytotoxicity.

FIG. 33A-FIG. 33B shows (FIG. 33A) Images and (FIG. 33B) number of CSC sphere formation after treatment (LM, LND and m104: 5 μM), scale bar: 200 μm (upper) and 100 μm (lower).

FIG. 34A-FIG. 34C shows (FIG. 34A) Viability curves of Stella cell. (FIG. 34B) Images and (FIG. 34C) diameter of CSC spheres (with Stella cell) after treatment (LM, LND and m104: 5 μM), scale bar: 100 μm.

FIG. 35A-FIG. 35B shows (FIG. 35A) Mitochondrial membrane potential analysis of CSC cells that were treated as indicated (5 μm) and stained with JC-1. scale bar: 10 μm. (FIG. 35B) Metabolic fluxes analysis of CSC cells treated as indicated were analyzed by tracing the oxygen consumption rates (OCRs) according to the Agilent Seahorse XF cell Mito Stress protocol of the manufacturer.

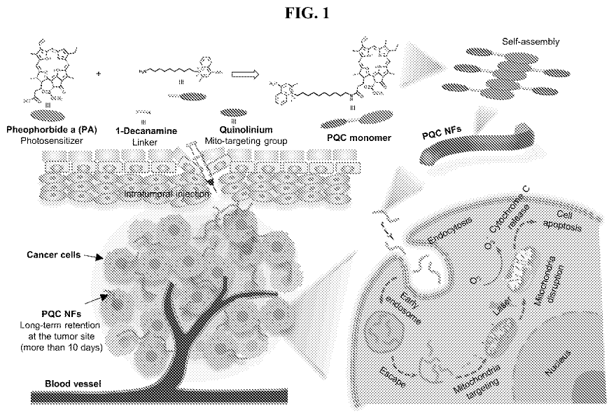

### DETAILED DESCRIPTION OF THE INVENTION

### I. General

The present invention provides photosensitizers of amphiphilic quinolinium-drug conjugates capable of self-assembly. The conjugates can form nanofibers and nanoparticles which exhibit significant phototoxicity to cancer cells by targeting mitochondria. The amphiphilic properties of the conjugates provided herein, allow for improved delivery to tumor sites and a greater inhibition of cancer cells. The conjugates have been found to achieve a 100% complete cure rate in both subcutaneous and orthotopic oral cancer models with only a single-dose administration.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₁₋₂₀, C₁₋₃₀, C₁₋₄₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 40 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₂₋₂₀, C₂₋₃₀, C₂₋₄₀, C₃, C₃₋₄, C₃₋₅, C₃₋₆, C₄, C_{4- 5}, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene. Alkenylene groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₂₋₂₀, C₂₋₃₀, C₂₋₄₀, C₃, C₃₋₄, C₃₋₅, C₃₋₆, C₄, C_{4- 5}, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, isopropynylene, butynylene, sec-butynylene, pentynylene and hexynylene. Alkynylene groups can be substituted or unsubstituted.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Polyethyleneglycol” refers to the polymer, with the following general structure:

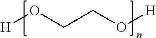

wherein the monomer may be substituted or unsubstituted, and wherein n is an integer equal to 5 or greater.

“Hydrophobic group” refers to a chemical moiety that is substantially water-insoluble. Examples of hydrophobic groups include, but are not limited to, long-chain alkanes and fatty acids, fluorocarbons, silicones, certain steroids such as cholesterol, and many polymers including, for example, polystyrene and polyisoprene.

“Hydrophilic group” refers to a chemical moiety that is substantially water-soluble. Examples of hydrophilic groups include, but are not limited to, alcohols, short-chain carboxylic acids, quaternary amines, sulfonates, phosphates, sugars, and certain polymers such as PEG.

“Amphiphilic compound” refers to a compound having both hydrophobic portions and hydrophilic portions.

“Photosensitizer” refers to compounds that can be activated by light in order to generate a reactive radical, typically a reactive oxygen species (ROS) for photodynamic therapy, but can also generate a reactive radical for polymerization, crosslinking, or degradation. Photosensitizers may be useful for treatment of diseases by producing singlet oxygen to damage tumors. Photosensitizers include, but are not limited to, porphyrins, dyes, and chlorophylls.

“Porphyrin” refers to any compound, with the following porphin core:

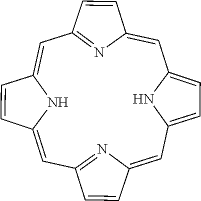 [SMILES: C1=Cc2cc3ccc(cc4nc(cc5ccc(cc1n2)[nH]5)C=C4)[nH]3]

wherein the porphin core can be substituted or unsubstituted.

“Terpene” or “terpenoid” refers to a class of organic compounds characterized by units of isoprene, which has the molecular formula C₅H₈. Non-limiting examples of terpenes include hemiterpenes, monoterpenes, sesquiterpenes, diterpenes, sesterterpenes, triterpenes, sequarterpenes, tetraterpenes, polyterpenes, and norisoprenoids. Triterpene” or “triterpenoid” refers to compounds composed of six isoprene units which are widely distributed in nature. Examples include ursolic acid and oleanolic acid (triterpenes), and sterols (triterpenoids).

“Nanofiber” refers to fibers having an average diameter not greater than about 1500 nanometers (nm). Nanofibers are generally understood to have a fiber diameter range of about 10 to about 1500 nm, more specifically from about 10 to about 1000 nm, more specifically still from about 20 to about 500 nm, and most specifically from about 20 to about 400 nm.

“Steroid” refers to any of a class of biomolecules that are responsible for a variety of biologically important functions such as signaling molecules. Examples of steroids include, but are not limited to, cholesterol, bile acids, sex hormones, and other synthetic drugs.

“Nanoparticle” refers to a micelle or liposomal structure resulting from aggregation or self-assembly of the compounds of the invention. The nanoparticles of the present invention can have a hydrophobic core and a hydrophilic exterior.

“Hydrophobic drug” or “therapeutic agent” refers to an agent capable of treating and/or ameliorating a condition or disease. A drug may be a hydrophobic drug, which is any drug that is substantially insoluble in water. Hydrophobic drugs useful in the present invention include, but are not limited to, indazole-3-carboxylic acid, lonidamine, tolnidamine, steroids, triterpenoids, botulin, β-lapachone, vitamin E, α-tocopheryl, α-tocopheryl succinate, or derivatives thereof. The drugs of the present invention also include prodrug forms. One of skill in the art will appreciate that other drugs are useful in the present invention.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Disease” or “disorder” refers abnormal cellular function in an organism, which is not due to a direct result of a physical or external injury. Diseases can refer to any condition that causes distress, dysfunction, disabilities, disorders, infections, pain, or even death. Diseases include, but are not limited to hereditary diseases such as genetic and non-genetic diseases, infectious diseases, non-infectious diseases such as cancer, deficiency diseases, and physiological diseases.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Target” or “targeting” refers to using a compound, protein, or antibody that specifically or preferentially binds to a cell, viral particle, viral protein, an antigen, or a biomolecule, or that is localized to a specific cell type, tissue type, microbe type, or viral type.

“Photodynamic therapy” refers to use of nontoxic, light-sensitive compounds that become toxic to malignant or disease cells upon exposure to light. Photodynamic therapy involves a photosensitizer, a light source, and oxygen. Upon exposure to the light, the photosensitizer generates reactive oxygen species (singlet oxygen, an oxygen free radical) that react with and destroy the malignant tissue. A variety of photosensitizers can be used, including porphyrins or a derivative thereof, chlorophylls and dyes.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

### II. Compounds

In some embodiments, the present invention provides a compound of Formula (I):

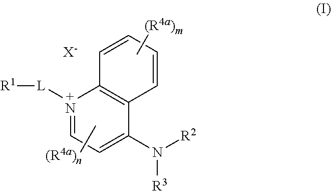

wherein: R¹ is a hydrophobic drug or photosensitizer; L is C₂₋₂₀ alkylene, C₂₋₂₀ alkenylene, C₂₋₂₀ alkynylene, or polyethyleneglycol polymer; R² is H, C₁₋₆ alkyl,

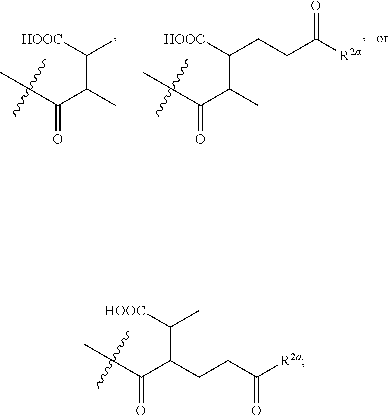

R^{2a} is —OH, —NH-polyethyleneglycol, polyethyleneglycol or a peptide; R³ is H or C₁₋₆ alkyl; each R^{4a} and R^{4b} is independently H, C₁₋₆ alkyl, C₂₋₆ alkenyl, C₂₋₆ alkynyl, halogen, or C₁₋₆ haloalkyl; subscript m is from 1 to 4; subscript n is from 1 to 2; and X is Cl, Br or I; wherein when R¹ is cyclosporin, R² is Me, and R³ is Me, then L is C₂₋₂₀ alkylene, C₁₀₋₂₀ alkenylene, C₂₋₂₀ alkynylene, or polyethyleneglycol polymer.

The hydrophobic drugs useful in the present invention can be any hydrophobic drug known by one of skill in the art. Hydrophobic drugs useful in the present invention include, but are not limited to, lonidamine, botulin, betulinic acid, β-lapachone, and α-tocopheryl succinate, pyrvinium, atovaquone, bedaquiline, antimycin A, oligomycin A, rotenone, piericidin A, Atpenin A5, 3-nitropropionic acid, myxothiazol, stigmatellin, aurovertin-B, and trifluoromethoxy carbonylcyanide phenylhydrazone.

In some embodiments, R¹ is the hydrophobic drug is indazole-3-carboxylic acid, lonidamine, tolnidamine, steroids, triterpenoids, botulin, β-lapachone, vitamin E, α-tocopheryl, α-tocopheryl succinate, or derivatives thereof. In some embodiments, the present invention provides compounds of Formula (I), wherein R¹ is lonidamine.

In some embodiments, R¹ is the photosensitizer. In some embodiments, the photosensitizer is a porphyrin. Any suitable porphyrin can be used for R¹ in the compounds of the present invention. Representative porphyrins suitable in the present invention include, but are not limited to, pyropheophorbide-a, pheophorbide, chlorin e6, purpurin or purpurinimide. In some embodiments, the porphyrin can be pyropheophorbide-a. Representative porphyrin structures are shown below:

| PORPHYRIN | STRUCTURE |
|---|---|
| Porphyrin | 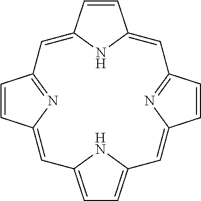 [SMILES: C1=Cc2cc3ccc(cc4nc(cc5ccc(cc1n2)[nH]5)C=C4)[nH]3] |
| Pyropheophorbide-a | 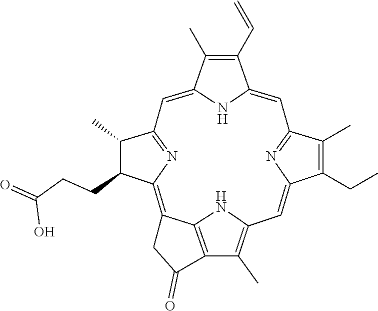 [SMILES: C=Cc1c(C)c2cc3nc(c4c5[nH]c(cc6nc(cc1[nH]2)C(C)=C6CC)c(C)c5C(=O)C4)[C@@H](CCC(=O)O)[C@@H]3C] |
| Pheophorbide | 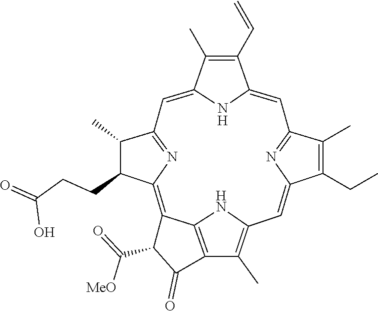 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)O)[C@@H]3C] |
| Chlorin e6 | 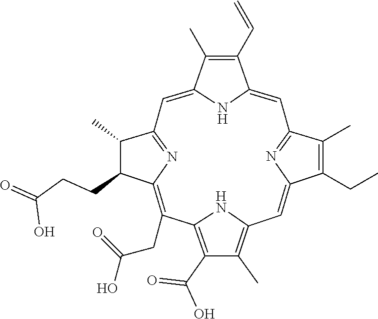 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C] |
| Purpurin | 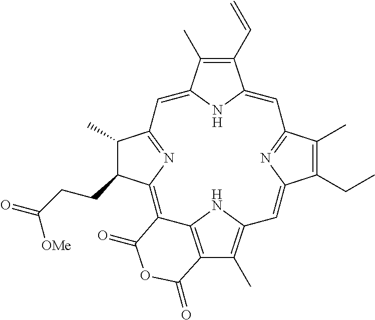 [SMILES: C=Cc1c(C)c2cc3nc(c4c5[nH]c(cc6nc(cc1[nH]2)C(C)=C6CC)c(C)c5C(=O)OC4=O)[C@@H](CCC(=O)OC)[C@@H]3C] |
| Purpurinimide | 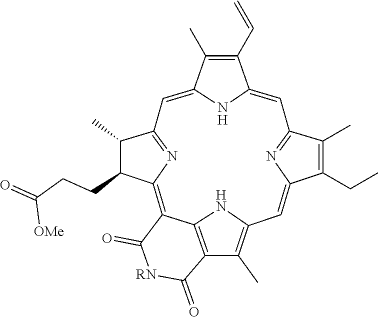 |

In some embodiments, photosynthesizer is pheophorbide.

In some embodiments, L is C₂₋₂₀ alkylene. L can be C₆₋₂₀ alkylene, C₈₋₁₆ alkylene, C₈₋₁₂ alkylene, or C₆ alkylene, C₈ alkylene, C₁₀ alkylene, C₁₂ alkylene, C₁₄ alkylene, or C₁₆ alkylene. In some embodiments, the present invention provides compounds of Formula (I), wherein L is C₁₀ alkylene.

In some embodiments, R² and R³ are each hydrogen.

In some embodiments, R^{4a} is H; and each R^{4b} is independently H or C₁₋₆ alkyl. In some embodiments, the present invention provides compounds of Formula (I), wherein each R^{4a} is H; and each R^{4b} is independently H or methyl.

In some embodiments, X is I⁻.

In some embodiments, R¹ is a hydrophobic drug or photosensitizer; L is C₂₋₂₀ alkylene; R² is H, or C₁₋₆ alkyl; R³ is H or C₁₋₆ alkyl; each R^{4a} and R^{4b} is independently H, or C₁₋₆ alkyl; subscript m is from 1 to 4; subscript n is from 1 to 2; and X is Cl, Br or I. In some embodiments, R¹ is a hydrophobic drug or photosensitizer; L is C₈₋₁₆ alkylene; R² is H, or C₁₋₆ alkyl; R³ is H or C₁₋₆ alkyl; R^{4b} is H, or C₁₋₆ alkyl; subscript n is 1; and X is Cl, Br or I. In some embodiments, R¹ is a hydrophobic drug or photosensitizer; L is C₁₀ alkylene; R² is H; R³ is H; R^{4b} is C₁₋₆ alkyl; subscript n is 1; and X is Cl, Br or I. In some embodiments, R¹ is a hydrophobic drug or photosensitizer; L is C₁₀ alkylene; R² is H; R³ is H; R^{4b} is methyl; subscript n is 1; and X is I.

In some embodiments, the compound has the structure:

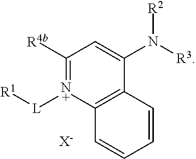

In some embodiments, the compound has the structure:

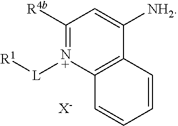

In some embodiments, the compound has the structure:

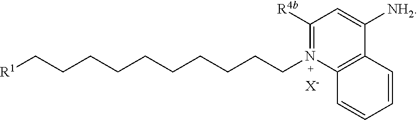

In some embodiments, the compound has the structure:

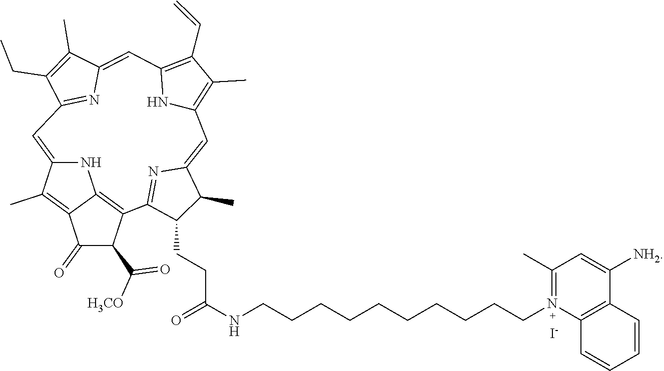

In some embodiments, the compound has the structure:

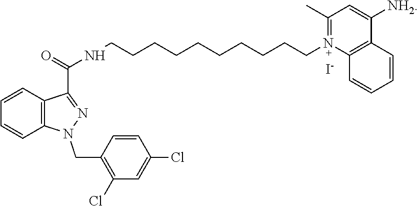

### IV. Nanofibers

In another embodiment, the present invention provides a nanofiber comprising a plurality of conjugates of Formula (I):

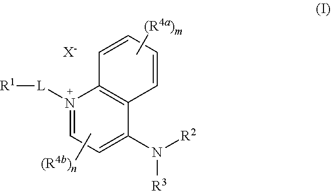

wherein: R¹ is a hydrophobic drug or photosensitizer; L is C₂₋₂₀ alkylene, C₂₋₂₀ alkenylene, C₂₋₂₀ alkynylene, or polyethyleneglycol polymer; R² is H, C₁₋₆ alkyl,

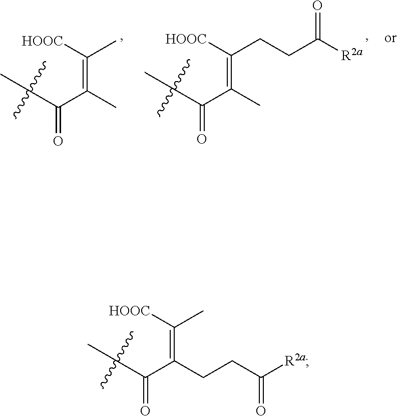

R^{2a} is —OH, —NH-polyethyleneglycol, polyethyleneglycol or a peptide; R³ is H or C₁₋₆ alkyl; each R^{4a} and R^{4b} is independently H, C₁₋₆ alkyl, C₂₋₆ alkenyl, C₂₋₆ alkynyl, halogen, or C₁₋₆ haloalkyl; subscript m is from 1 to 4; subscript n is from 1 to 2; and X is Cl, Br or I.

In some embodiments, each conjugate of Formula (I) is the compound:

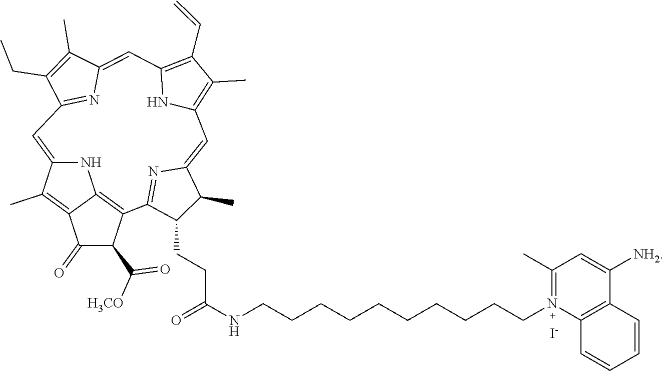

In some embodiments, each conjugate of Formula (I) is the compound:

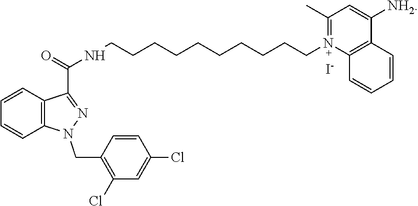

The nanofiber of the present invention can be used for cell or lysosomal targeting. The nanofiber can target the cell or lysosome to inhibit autophagy. In some embodiments, the nanofibers can target lysosomal disruption, lysosomal dysfunctional, autophagy inhibition, or a combination thereof. In some embodiments, the nanofiber target the lysosome.

In some embodiments, the nanofibers can accumulate in lysosomes. The formed nanofiber in lysosomes can cause lysosomal dysfunction and trigger apoptosis of cancer cells. Since containing the photosensitization group in the structure, this nanofibers of the present invention also support a highly effective lysosome-based photodynamic treatment that can intrinsically overcome the autophagy-associated drug resistance.

In some embodiments, the nanoparticles comprises a plurality of compounds of the present invention, with the compound structures as described above.

The nanofibers (NFs) of the present invention can be prepared by a variety of methods, such as from the pheophorbide a (PA) and quinolinium conjugate (PQC) monomer. The PQC NFs can be prepared from the PQC monomers by adding deionized water added dropwise to an ethanol solution. Ethanol can then be removed from the solution by rotary evaporation at 37° C. wherein, the nanofibers are formed spontaneously.

### V. Nanoparticles

In another embodiment, the present invention provides a nanoparticle comprising a plurality of conjugates of Formula (I):

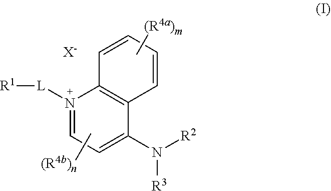

wherein: R¹ is a hydrophobic drug or photosensitizer; L is C₂₋₂₀ alkylene, C₂₋₂₀ alkenylene, C₂₋₂₀ alkynylene, or polyethyleneglycol polymer; R² is H, C₁₋₆ alkyl,

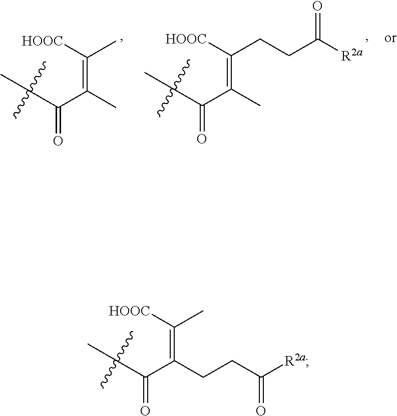

R^{2a} is —OH, —NH-polyethyleneglycol, polyethyleneglycol or a peptide; R³ is H or C₁₋₆ alkyl; each R^{4a} and R^{4b} is independently H, C₁₋₆ alkyl, C₂₋₆ alkenyl, C₂₋₆ alkynyl, halogen, or C₁₋₆ haloalkyl; subscript m is from 1 to 4; subscript n is from 1 to 2; and X is Cl, Br or I.

In some embodiments, each conjugate is the compound:

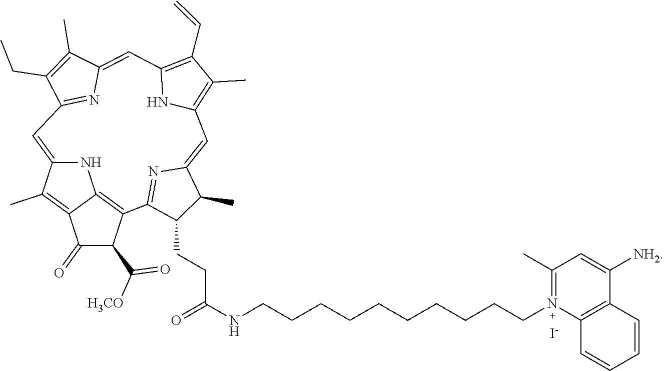

In some embodiments, each conjugate is the compound:

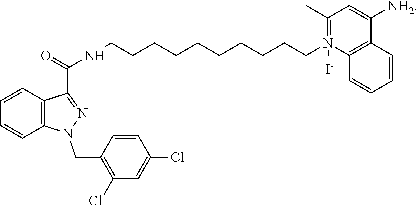

In some embodiments, liposomes are formed when phospholipids and their derivatives are dispersed in water, wherein the phospholipids form closed vesicles called “liposomes”. A wide variety of liposomes have been used as carriers for entrapped therapeutic agents, such as drugs, enzymes, and genetic sequences for use in medical science, in pharmaceutical science and in biochemistry.

In some embodiments, the membrane constituents of the nanoparticles of the present invention include phospholipids and/or phospholipid derivatives. Representative phospholipids and phospholipid derivatives, include, but are not limited to, phosphatidyl ethanolamine, phosphatidyl choline, phosphatidyl serine, phosphatidyl inositol, phosphatidyl glycerol, cardiolipin, sphingomyelin, ceramide phosphorylethanolamine, ceramide phosphoryl glycerol, ceramide phosphoryl glycerol phosphate, 1,2-dimyristoyl-1,2-deoxyphosphatidyl choline, plasmalogen, phosphatidic acid, etc. One or more phospholipids can be used in the nanoparticles of the present invention.

In some embodiments, the nanoparticles and lipid nanoparticles of the present invention can contain any suitable lipid. Representative lipids include, but are not limited to, cationic lipids, zwitterionic lipids, neutral lipids, or anionic lipids as described above. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

In some embodiments, the phospholipids can include phosphatidylcholine (PC), phosphatidic acid (PA), phosphatidylethanolamine (PE), phosphatidylglycerol (PG), phosphatidylserine (PS), and phosphatidylinositol (P1), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. Lipid extracts, such as egg PC, heart extract, brain extract, liver extract, and soy PC, are also useful in the present invention. In some embodiments, soy PC can include Hydro Soy PC (HSPC). In some embodiments, the lipids can include derivatized lipids, such as PEGylated lipids. Derivatized lipids can include, for example, DSPE-PEG2000, cholesterol-PEG2000, DSPE-polyglycerol, or other derivatives generally known in the art.

In some embodiments, liposomes and nanoparticles of the present invention may contain steroids. Representative steroids can be characterized by the presence of a fused, tetracyclic gonane ring system. Examples of steroids include, but are not limited to, cholesterol, cholic acid, progesterone, cortisone, aldosterone, estradiol, testosterone, dehydroepiandrosterone. Synthetic steroids and derivatives thereof are also contemplated for use in the present invention.

In some embodiments, the liposome or nanoparticle can include one or more lipids which can be a phospholipid, a steroid, and/or a cationic lipid. In some embodiments, the phospholipid is a phosphatidylcholine, a phosphatidylglycerol, a phosphatidylethanolamine, a phosphatidylserine, a phosphatidylinositol, or a phosphatidic acid. In some embodiments, the phosphatidylcholine is DSPC. In some embodiments, the phosphatidylglycerol is DSPG. In some embodiments, the phosphatidylethanolamine is DSPE-PEG(2000). In some embodiments, the steroid is cholesterol.

In some embodiments, the liposome or nanoparticle can include monosaccharides such as glucose, galactose, mannose, fructose, inositol, ribose, and xylose; disaccharides such as lactose, sucrose, cellobiose, trehalose, and maltose; trisaccharides such as raffinose and melizitose; polysaccharides such as cyclodextrin; and sugar alcohols such as erythritol, xylitol, sortibol, mannitol and maltitol; polyvalent alcohols such as glycerin, diglycerin, polyglycerin, propylene glycol, polypropylene glycol, ethylene glycol, diethylene glycol, triethylene glycol, polyethylene glycol, ethylene glycol monoalkylether, diethylene glycol monoalkylether, 1,3-butylene glycol. Combinations of sugar and alcohol can also be used.

In some embodiments, the nanoparticle comprises 1-alpha-phosphatidylcholine, cholesterol, and mPEG-DSPE.

In some embodiments, the nanoparticles comprises a plurality of compounds of the present invention, with the compound structures as described above.

The nanoparticles of the present invention can be prepared by a variety of methods. For example, the nanoparticles can be prepared using a thin-film hydration method. The method involves adding L-α-phosphatidylcholine, cholesterol, mPEG-DSPE, and PQC or pheophorbide a to chloroform to dissolve. The chloroform solution can then be evaporated to form a thin film and a phosphate buffered saline (PBS) buffer can be added to re-hydrate the thin film.

### VI. Formulations and Administration

The compounds, nanofibers, nanoparticles, and compositions of the present invention can be prepared in a wide variety of oral, parenteral and topical dosage forms. Oral preparations include tablets, pills, powder, dragee, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds, nanofibers, and nanoparticles of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds, nanofibers, and nanoparticles of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### VI. Method of Treatment

In some embodiments, the present invention provides a method of treating a disease, the method comprising administering a therapeutically effective amount of a nanofiber of Formula (I) or a nanoparticle of Formula (I) to a subject in need thereof.

In some embodiments, the method further comprises combination therapy by using additional agents for treating the disease. The additional agent is a therapeutic agent. Combination therapy of the present invention includes, but is not limited to, using a nanofiber or nanoparticle of the present invention, and one or more additional agent.

Combination therapy can include, but is not limited to immunotherapy, radiation therapy, chemotherapy, molecular targeted therapy, or a combination thereof.

In some embodiments, the method further comprises one or more additional agents, wherein the additional agent is a chemotherapeutic agent, a molecular targeted agent, an immunotherapeutic agent, a radiotherapeutic agent or a combination thereof. In some embodiments, the additional agent is the immunotherapeutic agent. Immunotherapeutic agents useful in the present invention are listed above. In some embodiments, the additional agent is the radiotherapeutic agent. Radiotherapeutic agents useful in the present invention are listed above. In some embodiments, the additional agent is the chemotherapeutic or molecular targeted agent. Chemotherapeutic and molecular targeted agents useful in the present invention are listed above.

In some embodiments, the one or more additional agents comprise two additional agents. In some embodiments, the additional agents are the immunotherapy agent and radiotherapeutic agent. In some embodiments, the additional agents are the immunotherapeutic agent and the chemotherapeutic agent. In some embodiments, the additional agents are the immunotherapeutic agent and molecular targeted agent. In some embodiments, the additional agents are the radiotherapeutic agent and chemotherapeutic agent. In some embodiments, the additional agents are the radiotherapeutic agent and molecular targeted agent.

In some embodiments, the additional agent is a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase (mek) inhibitor, a VEGF trap antibody, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR; INO 1001, IPdRT KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated, estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(But) 6, Azgly 10](pyro-Glu-His-Trp-Ser-Tyr-D-Ser(But)-Leu-Arg-Pro-Azgly-NH2 acetate [C₅₉H₈₄N₁₈O₁₄—(C₂H₄O₂)X where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifamib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilumumab, vemurafenib, or a combination thereof. In some embodiments, the additional agent is HCQ, Lys05, JQ1, rapamycin, napabucasin, ipilimumab, nivolumab, pembrolizumab, atezolizumab, avelumab, durvalumab, β-lapachone, cisplatin, nimorazole, cetuximab, misonidazole, tirapazamine, daunorubicin, doxorubicin, paclitaxel, docetaxel, abraxane, bortezomib, etoposide, lenalidomide, apoptozole, carboplatin, cisplatin, oxaliplatin, vinblastine, vincristine, trastuzumab, erlotinib, imatinib, nilotinib, vemurafenib, or a combination thereof.

The nanofibers and nanoparticles of the present invention can be administered to a subject for treatment, e.g., of hyperproliferative disorders including cancer such as, but not limited to: carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, CANCER: PRINCIPLES AND PRACTICE (DeVita, V. T. et al. eds 2008) for additional cancers).

Diseases treated by the method of the present invention includes coronavirus, malaria, antiphospholipid antibody syndrome, lupus, rheumatiod arthritis, chronic urticaria or Sjogren's disease and cancer such as, but not limited to: carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, CANCER: PRINCIPLES AND PRACTICE (DeVita, V. T. et al. eds 2008) for additional cancers).

In some embodiments, the disease is cancer. In some embodiments, the cancer is bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer, prostate and uterine cancer. In some embodiments, the cancer is bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer and uterine cancer. In some embodiments, the disease is oral squamous cell carcinoma, pancreatic cancer, bladder cancer, or glioma. In some embodiments, the disease is oral squamous cell carcinoma. In some embodiments, the disease is pancreatic cancer. In some embodiments, the disease is glioma.

In some embodiments, the method of treating the disease comprises targeting cell autophagy and/or the lysosome. Targeting autophagy can result in either autophagy inhibition or autophagy activation. Targeting the lysosome can result in lysosomal disruption, lysosomal dysfunction, or both.

In some embodiments, the method of treating targets lysosomal disruption, lysosomal dysfunction and/or autophagy inhibition. In some embodiments, the method of treating targets the lysosome.

In some embodiments, the nanocarrier targets lysosomal disruption, lysosomal dysfunction and/or autophagy inhibition. In some embodiments, the nanocarrier targets the lysosome.

In some embodiments, the present invention provides a method of treating a disease via photodynamic therapy, the method comprising administering a therapeutically effective amount of a nanofiber of Formula (I) or a nanoparticle of Formula (I), wherein R¹ is a photosensitizer, to a subject in need thereof.

The methods of treating using the nanofibers and nanoparticles of the present invention also includes treating a disease by photodynamic therapy or photothermal therapy.

The methods generally involve administering a nanofiber or nanoparticle of the present invention to a subject, and then exposing the subject to radiation of a specific wavelength to induce the photodynamic or photothermal therapy depending on the wavelength of light. Upon exposure to the radiation or light, the porphyrins used in the nanofibers and nanoparticles of the present invention, either complexed to a metal or not, generate either the reactive singlet oxygen suitable for photodynamic therapy, or generate heat sufficient of photothermal therapy. In some embodiments, the present invention provides a method of treating a disease via photodynamic or photothermal therapy, including administering to a subject in need thereof, a therapeutically effective amount of a nanofiber or nanoparticle of the present invention, and exposing the subject to radiation, thereby treating the disease via photodynamic or photothermal therapy. In some embodiments, the method is a method of treating a disease via photodynamic therapy. In other embodiments, the method is a method of treating a disease via photothermal therapy.

In some embodiments, the present invention provides a method of treating a disease via photodynamic or photothermal therapy, including administering to a subject in need thereof, a therapeutically effective amount of a nanofiber or nanoparticle of the present invention, and optionally a drug (e.g., inhibitor of vascularization), and exposing the subject to electromagnetic radiation, thereby treating the disease via photodynamic or photothermal therapy. In some embodiments, the method is a method of treating a disease via photodynamic therapy. In other embodiments, the method is a method of treating a disease via photothermal therapy. In some cases, the electromagnetic radiation has a controlled wavelength. In some cases, the vascular abnormality is exposed to electromagnetic radiation from a laser, such as a diode laser (e.g., a 405 nm diode laser). In some cases, the vascular abnormality is exposed to electromagnetic radiation from a light emitting diode (e.g., a 410 nm light emitting diode). In some cases, the electromagnetic radiation has or contains photons having a wavelength of about 405 nm (e.g., between about 400 and about 420 nm) or about 680 nm (e.g., between about 600 and about 700), or a combination thereof.

In some embodiments, the disease treated by the method of the present invention is a cancer. In some embodiments, the disease is oral squamous cell carcinoma.

In some embodiments, the method of the present invention comprises a conjugate of Formula (I), wherein the nanofiber is the compound:

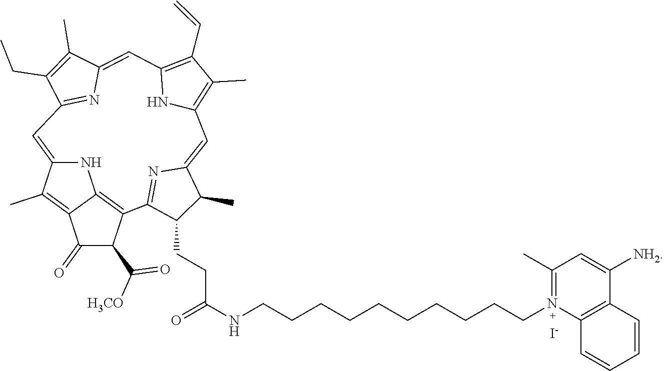

### VIII. EXAMPLES

### Example 1. Compounds

Preparation and Characterization of PQC NFs: Briefly, PQC monomers in ethanol were added dropwise into deionized water under stirring. Ethanol in the solution was removed by vacuum rotary evaporation at 37° C. and the self-assembling PQC NFs were formed spontaneously. The aqueous nanofiber solution (0.02 mg mL⁻¹) was deposited on copper grids to prepare samples for transmission electron microscope (TEM). The morphology was observed by a Talos L120C TEM (Thermo Fisher Scientific, USA). The UV-Vis and fluorescence spectra were measured by a UV-Vis spectrometer (UV-1800, Shimadzu, Japan) and a fluorescence spectrometer (RF-6000, Shimadzu, Japan), respectively. NIR fluorescence imaging studies was performed by a ChemiDoc™ MP imaging system (Bio-Rad, USA). The ¹O₂ production was detected using SOSG (Thermo Fisher Scientific, USA) as an indicator. Briefly, SOSG solution was added into drug solutions in 96 wells plate. The mixed solutions containing 0.25% SDS (w/v) or not were then irradiated for 60 s using a 633-nm LED array (Omnilux new-U, PhotoTherapeutics, USA) at a power density of 30 mW cm⁻² at room temperature. Fluorescence intensity was determined by a microplate reader (Tecan, Switzerland). The ultrafiltration experiment of PQC NFs were conducted using a centrifuge (10k rpm, 10 min) and a centrifuge tube (10 kDa, Beckman Coulter, USA).

### Chemicals and Instruments

Pheophorbide a was purchased from Santa Cruz Biotechnology (TX, USA). 1,10-Diiododecane, 4-aminoquinaldine, Phthalimide potassium salt, hydrazine, 2-butanone, and N-(3-dimethylaminopropyl)-N-ethylcarbodiimide (EDC) hydrochloride were purchased from Millipore-Sigma (MO, USA). 6-Chloro-1-hydroxybenzotriazole (6-C-HOBT) and N,N-diisopropylethylamine (DIEA) were obtained from Chem-Impex International, Inc (IL, USA).

Anhydrous sodium sulfate, sodium chloride and organic solvents were purchased from Fisher Scientific (MA, USA). All solvents were used directly without further purification. Water used in experiments was purified with a Mill-Q filtration system. NMR spectra were collected by a 600 MHz NMR spectrometer (Bruker, German). Mass Spectra were recorded by an LTQ-Orbitrap XL Hybrid ion trap mass spectrometer (ESI model, Thermo Fisher, USA) or a UltraFlextreme MALDI TOF/TOF mass spectrometer (Bruker, German).

### Synthesis of Compound 1

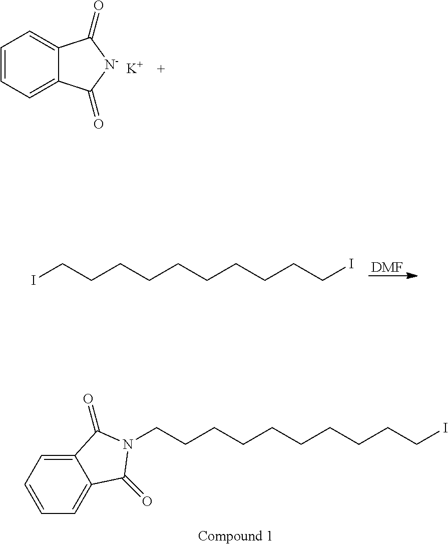

To the solution of 1,10-diiododecane (1.18 g, 3.0 mmol, 3.0 eq) in 25 mL dimethylformamide was added Phthalimide potassium salt (158 mg, 1.0 mmol, 1.0 eq). The mixture was stirred for 24 h at room temperature. The mixture was diluted with 100 mL ethyl acetate, washed with water and brine, dried over with anhydrous sodium sulfate. After filtration and concentration, the crude product was purified via silica column to obtain compound 1.1 Yield: 360 mg, 88.3%. ¹HNMR (DMSO-d₆, 600 MHz) δ 7.88 (m, 4H), 3.57 (t, 2H, J=6.6 Hz), 3.27 (t, 2H, J=7.2 Hz), 1.74 (m, 2H), 1.59 (m, 2H), 1.31 (m, 12H); ESI-MS found [M+H]⁺ 414.0927.

### Synthesis of Compound 2

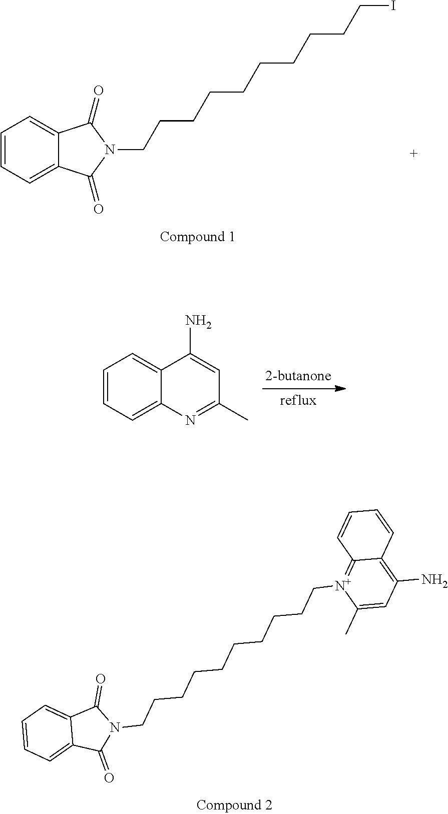 [SMILES: O=C1c2ccccc2C(=O)N1CCCCCCCCCCI ; Cc1cc(N)c2ccccc2n1 ; Cc1cc(N)c2ccccc2[n+]1CCCCCCCCCCN1C(=O)c2ccccc2C1=O]

The solution of compound 1 (1.24 g, 3 mmol, 3.0 eq) and 4-aminoquinaldine (158.2 mg, 1 mmol, 1.0 eq) in 2-butanone (30 mL) was refluxed at 95° C. for 4 d. The crude produce was purified via silica column to afford compound 2. Yield: 175 mg, 39.4%. ¹HNMR (DMSO-d₆, 600 MHz) δ 8.84 (d, 2H, J=15.6 Hz), 8.47 (d, 1H, J=8.4 Hz), 8.16 (d, 1H, J=9.0 Hz), 8.03 (t, 1H, J=7.8 Hz), 7.87 (m, 4H), 7.74 (t, 1H, J=7.8 Hz), 6.72 (s, 1H), 4.46 (t, 2H, J=7.2 Hz), 3.57 (t, 2H, J=7.2 Hz), 2.73 (s, 3H), 1.72 (m, 2H), 1.59 (m, 2H), 1.44 (m, 2H), 1.32 (m, 10H); ESI-MS found [M−I]⁺ 444.2633.

### Synthesis of Compound 3

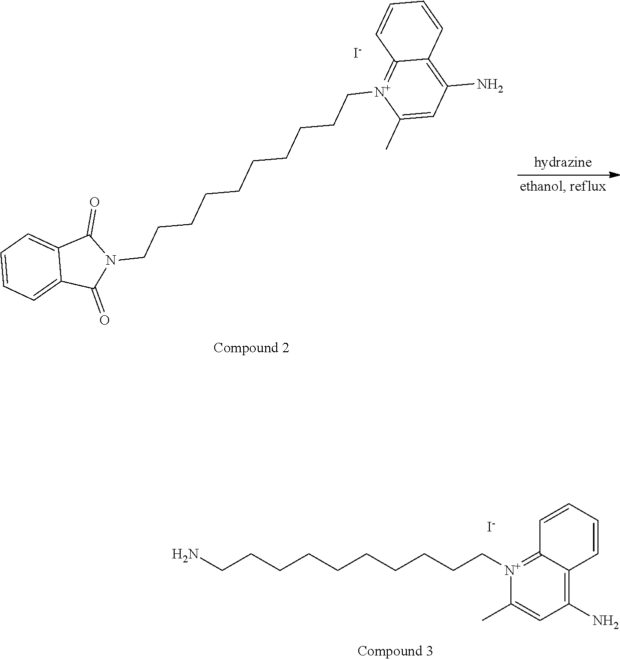 [SMILES: Cc1cc(N)c2ccccc2[n+]1CCCCCCCCCCN1C(=O)c2ccccc2C1=O ; [I-] ; Cc1cc(N)c2ccccc2[n+]1CCCCCCCCCCN ; [I-]]

Compound 2 (285.8 mg, 0.5 mmol, 1.0 eq) and hydrazine (62.8 mg, 2.0 mmol, 4.0 eq) were dissolved in 20 mL ethanol and refluxed for 12 h at 90° C. The mixture was extracted with ethyl acetate and washed with 1M NaOH three times. The organic phase was collected and dried by anhydrous sodium sulfate overnight. The solvent was removed to afford compound 3 as an oil. Yield: 88.5 mg, 40.1%. ¹HNMR (DMSO-d₆, 600 MHz) δ 9.09 (d, 2H, J=28.8 Hz), 8.57 (dd, 1H, J₁=8.4 Hz, J₂=1.2 Hz), 8.17 (d, 1H, J=9.0 Hz), 8.05 (broad, 2H), 8.03 (t, 1H, J₁=7.2 Hz, J₂=1.2 Hz), 7.72 (t, 1H, J=7.8 Hz), 6.79 (s, 1H), 4.47 (t, 2H, J=8.4 Hz), 2.73 (m, 5H), 1.74 (m, 2H), 1.58 (m, 2H), 1.48 (m, 2H), 1.32 (m, 10H); ESI-MS found [M−I]⁺ 414.2593.

### Synthesis of PQC Monomer

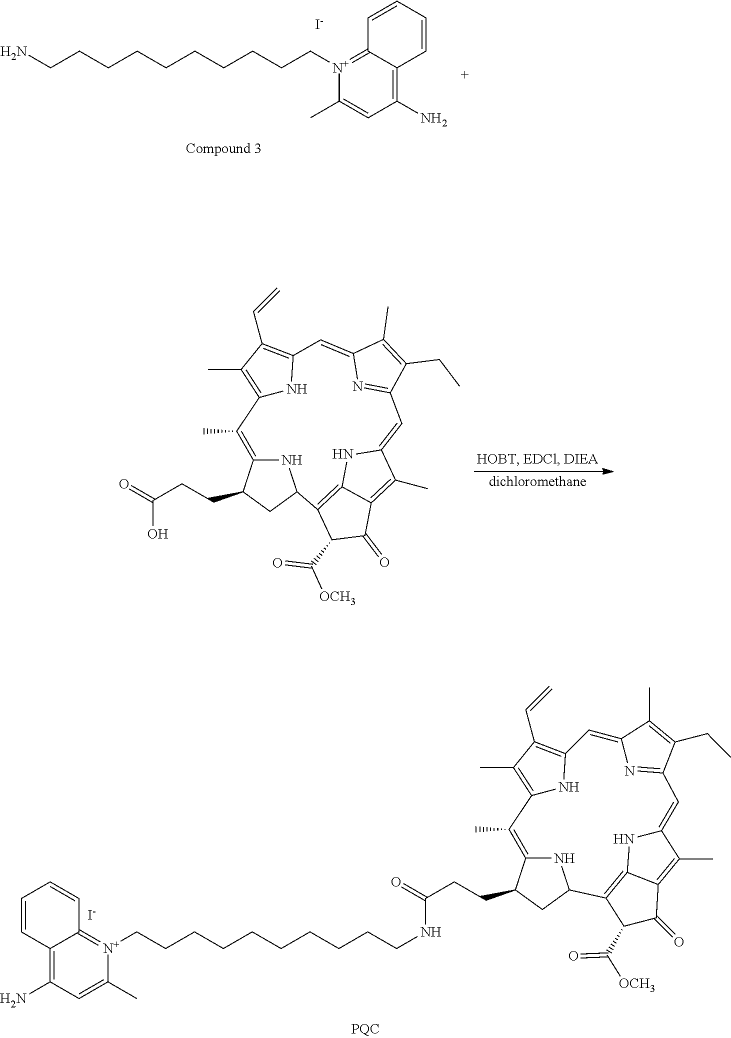

The solution of pheophorbide a (59.3 mg, 0.1 mmol, 1.0 eq), 6-chloro-1-hydroxybenzotriazole (203.5 mg, 0.12 mmol, 1.2 eq) and ethylcarbodiimide hydrochloride (230 mg, 0.12 mmol, 1.2 eq) in 15 mL dichloromethane was stirred at room temperature for 30 min. After compound 3 (85.8 mg, 0.15 mmol, 1.5 eq) and DIEA (35 μL, 0.2 mmol, 2 eq) were added, the mixture was vigorously stirred at room temperature for 8 h. The crude product was purified via silica column to obtain PQC monomer. Yield: 41 mg, 40.3%. ¹HNMR (DMSO-d₆, 600 MHz) δ 9.69 (s, 1H), 9.35 (s, 1H), 8.90 (s, 1H), 8.86 (broad, 2H), 8.38 (dd, 1H, J₁=8.4 Hz, J₂=1.2 Hz), 8.19 (m, 1H), 7.83 (t, 1H, J₁=8.4 Hz, J₂=1.2 Hz), 7.69 (d, 1H, J=9.0 Hz), 7.62 (m, 2H), 6.55 (s, 1H), 6.42 (s, 1H), 6.39 (dd, 1H, J₁=18.6 Hz, J₁=0.6 Hz), 6.22 (dd, 1H, J₁=12.0 Hz, J₁=0.6 Hz), 4.59 (m, 1H), 4.04 (s, 1H), 3.83 (m, 5H), 3.65 (m, 5H), 3.42 (s, 3H), 3.16 (s, 3H), 2.88 (m, 2H), 2.36 (s, 3H), 2.26 (m, 2H), 2.10 (m, 1H), 1.79 (d, 3H, J=7.8 Hz), 1.62 (m, 4H), 1.39 (m, 2H), 1.13 (m, 16H); ESI-MS found [M−I]⁺ 888.5101.

### Example 2. Biological Assays

### Cell Viability Assay

OSC-3, BXPC-3, AsPC-1, PANC-1, UM-UC-3, 5637, or IMR90 cells were seeded in 96-well plates at a density of 5×10³ cells per well and were grown overnight. Cells were incubated with various concentrations of drugs for 24 h, followed washing with PBS three times, and adding 100 μL fresh medium. For light-treated groups, cells were irradiated for 30 s using a 633-nm LED array (Omnilux new-U) at a power density of 30 mW cm⁻² at room temperature and then were cultured for another 24 h in parallel with non-light treated groups. Cell viability was quantified using the CellTiter-Glo assay (Promega, USA).

### Cellular Uptake Assay

OSC-3 were seeded in 96-well plate at a density of 5×10³ cells per well and grown overnight. PA and PQC NFs (1 μM) were added at predetermined time points. At the end, cells were lysed for 15 min with DMSO solution containing 0.5% Triton x-100. Fluorescence intensity (Ex=412 nm, Em=675 nm) was measured by a microplate reader (Tecan, Switzerland).

### Endocytosis Pathway Study

OSC-3 cells were seeded in 6-well plate (3×10⁵ cells per well) and were grown overnight. Cells were subjected to various treatments as follow: (1) 30 min incubation at either 4° C. or 37° C.; (2) 60 min incubation with endocytosis inhibitors: sodium azide (1 mg mL⁻¹, Sigma-Aldrich), chlorpromazine (20 μg mL⁻¹, Sigma-Aldrich), genistein (10 μg mL⁻¹, Combi-Blocks) and amiloride (50 μM, Alfa Aesar). Cells were then treated with PQC NFs (1 μM) for 2 h. After washed three times with cold PBS, cells were lysed for 15 min with DMSO solution containing 0.5% Triton x-100. Fluorescence intensity (Ex=412 nm, Em=675 nm) was measured by a microplate reader (Tecan, Switzerland).

### Colocalization Assay

OSC-3 cells were treated with PQC NFs (2 μM) for various times (from 5 min to 8 h) or with PA (2 μM) for 4 h, followed by incubation with LysoTracker Green (Thermo Fisher Scientific, USA) and MitoTracker Red (Cell Signaling Technology, USA) for 30 min. Cells were visualized on confocal laser scanning microscopy (CLSM) (Carl Zeiss, Germany) immediately to investigate the subcellular localization. Signals of PQC NPs and PA were observed on the Cy5 channel. LysoTracker and MitoTracker were observed according the manufacturer's instructions. The corresponding Pearson correlation coefficient was calculated by ImageJ software.

### Isolation of Mitochondrial, Lysosomal, and Cytoplasmic Fractions

2×10⁷ OSC-3 cells were seeded into a 150 mm cell culture dishes and grown overnight. Cells were treated for 24 h with PQC NFs or PA (1 μM), respectively. After washing with PBS three times, the mitochondrial and cytoplasmic fractions were isolated using a mitochondria isolation kit (Thermo Fisher Scientific, USA), or the lysosomes were isolated using a lysosome Enrichment Kit (Thermo Fisher Scientific, USA). Fluorescence intensity of the mitochondrial and cytoplasm fractions were determined by a microplate reader (Tecan, Switzerland). Data were normalized by per μg protein.

### Measurement of Intracellular ROS Production

OSC-3 cells were seeded in 6-well plates at a density of 5×10⁵ cells per well and were grown overnight. Cells were then were treated with PA and PQC NFs (1 μM) for 24 h. After washed three times with PBS, cells were incubated with 10 μM of 2′,7′-dichlorofluorescein diacetate (DCF-DA) (Sigma-Aldrich, USA) for 20 min, followed by another three times washing procedure with PBS. Cells were irradiated for 30 s using a 633-nm LED array (Omnilux new-U) at a power density of 30 mW cm⁻² at room temperature and then were cultured for 30 min. Cells were collected and analyzed by a BD FACSCanto flow cytometer (BD, USA).

### Mitochondrial Membrane Potential Assay

The mitochondrial membrane potential was determined using the dye JC-1 as a probe (Thermo Fisher Scientific, USA). Briefly, OSC-3 cells (2×10⁴ cells per well) were treated with drugs (1 μM) for 24 h. After washing three times with PBS and adding 100 μL fresh medium, cells were irradiated for 30 s using a 633-nm LED array (Omnilux new-U) at a power density of 30 mW cm⁻² at room temperature and were cultured for another 2 h. JC-1 (5 μg mL⁻¹) was added to incubate for 20 min. Cell imaging was performed on a CLSM (Carl Zeiss, Germany). The ratio of red/green fluorescence intensity was calculated by ImageJ software.

### Apoptosis Assay

Apoptosis assay was performed with the Annexin V-APC/propidium iodide (PI) apoptosis kit (Biolegend, USA). Briefly, OSC-3 cells (5×10⁵ cells per well) were treated with different drugs (0.2 μM) for 24 h. After washing three times with PBS and adding 100 μL fresh medium, cells were irradiated for 30 s using a 633-nm LED array (Omnilux new-U) at a power density of 30 mW cm⁻² at room temperature, and were cultured for another 12 h. Cells were stained with the apoptosis kit according to the manufacturer's instructions. All samples of cells were collected for flow cytometry using a BD FACSCanto flow cytometer (BD, USA). Data analysis was accomplished using FlowJo software.

### Western Blot Analysis

OSC-3 cells were treated with PQC NFs (0.5 μM) for 24 h, followed by washing three times with PBS and treatment with or without light (30 mW cm⁻²) for 30 s. Cells were cultured for another 24 h, and then the mitochondrial and the cytoplasmic proteins were isolated using a mitochondria isolation kit (Thermo Fisher Scientific, USA). Proteins were quantified using a BCA protein assay kit (Thermo Fisher Scientific, USA), then separated on 12% SDS-polyacrylamide gel electrophoresis (PAGE), and finally transferred onto polyvinylidene difluoride (PVDF) membranes (Millipore Sigma, USA). The membrane was blocked by 5% non-fat milk for 1 h and then incubated with the primary antibodies at 4° C. overnight. After subsequent washing with tris-buffered saline with 0.1% Tween 20 (TBST), the membrane was incubated with the secondary antibody for 1 h at room temperature. The immunoreactive bands were detected using the enhanced chemiluminescence detection kit (ProtoGlow ECL, National Diagnostics, USA) and imaged by the ChemiDoc™ MP imaging system (Bio-Rad, USA). Antibodies were used as followed: cytochrome C, caspase-3, cleaved caspase-3, PARP, and β-actin. All antibodies were from Cell Signaling Technology (USA).

### Cell and Tissue TEM Study

OSC-3 cells (20k per well) in an 8-well slide plate (Thermo Fisher Scientific, USA) were incubated with drugs (0.5 μM) for 24 h, then were washed with PBS three times, and were treated with or without light for 30 s. After another 2 h incubation, cells were fixed with the 0.1 M cacodylate buffer containing 2.5% glutaraldehyde plus 2% paraformaldehyde, and transferred to the carbon square mesh, followed by observation using a Talos L120C TEM (Thermo Fisher Scientific, USA). In the investigation of intracellular dissociation, PQC NFs (100 μM) were incubated with the freshly isolated mitochondria (0.1 mg mL⁻¹), lysosomes (0.1 mg mL⁻¹), and other cellular components (0.1 mg mL⁻¹) for 24 h and were then observed by the same TEM as mentioned above.

### Animal Models

Female athymic nude mice, 6-week-old, were purchased from Envigo (Indianapolis, IN, USA). All animal experiments were strictly performed in compliance with the protocol (#20265) approved by the Institutional Animal Care and Use Committee at the University of California, Davis. The subcutaneous and orthotopic tumor models were established by inoculated OSC-3 cells into both flanks (5×10⁶ cells per tumor) or lower lips (1×10⁶ cells per tumor) of nude mice. When the subcutaneous tumors reached about 80 mm³ and orthotopic tumors reached about 50 mm³, the mice started to be treated as indicated.

### In Vivo Imaging Study

The subcutaneous tumor model was used in fluorescence imaging study in vivo of PA and PQC NFs. PA and PQC NFs (10 nmol per 50 mm³ tumor) were administered by intratumoral injection. Fluorescence imaging were performed on a ChemiDoc™ MP imaging system (Bio-Rad, USA) at different time points post-injection.

### In Vivo Anticancer Study

Laser (680 nm) treatment was conduct by a laser device within an energy of 0.2 W cm⁻² for 6 min (Shanghai Xilong Optoelectronics Technology, China). Mice bearing subcutaneous and orthotopic tumors were randomly divided into four groups (n=6): Vehicle (PBS), DQA, PA, and PQC NFs. The drug was intratumorally injected into mice at a dose of 1 mM (10 nmol per 50 mm³ tumor). Laser treatment was performed for a total of four times, one each on days 1, 2, 5, and 6 post-injection of drugs. The relative tumor value and body weight were recorded every two days. In subcutaneous models, the tumors in the right flank of mice were subjected to laser treatment, while the left tumors were not. In orthotopic models, all tumors were treated with the laser treatment. The temperature of tumor surface was monitored using a thermal camera (FLIR Systems, USA) immediately after laser treatment. ROS production in the tumor after light treatment was also measured using DCF-DA as an indicator. ROS production in the tumor after light treatment was also measured using DCF-DA as an indicator. Briefly, the tumor was treated with different drugs via intratumoral injection at first. Then, the mice with tumors were treated with the laser at 1st, 2nd, 5th, and 6th day independently. After each light treatment, the mice were sacrificed immediately, and the obtained tumors were cut into small pieces and immersed in the DCF-DA solutions for 10 min. Finally, the tumors pieces were imaged by the ChemiDoc™ MP imaging system with the FITC channel. The fluorescence intensity of DCF-DA was quantified by Image J software. Mice were sacrificed after the first light treatment to obtain tumor and organs tissue for TEM, H&E, and IHC evaluation.

### Result and Discussion

### Synthesis and Characterization of PQC Monomer

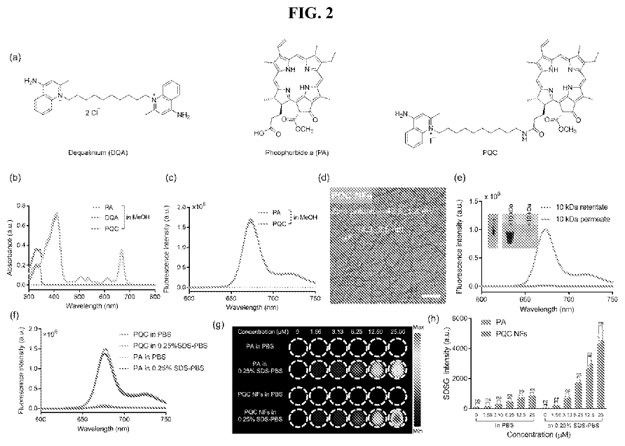

The PQC monomer was synthesized by conjugating PA with 4-aminoquinaldine through a 1-decanamine linker (FIG. 6). All intermediates and the target compound were chemically characterized by nuclear magnetic resonance (NMR) spectroscopy and electrospray-ionization mass spectrometry (MS). The UV-visible and fluorescence spectra were also used for structure confirmation (FIG. 2a). Together with the PA and PQC molecules, DQA, was also employed as the control. Free PQC molecules showed three main absorption peaks, with one at ˜350 nm from the absorbance of quinolinium moiety and the other two at 412 nm and 675 nm from the absorbance of PA (FIG. 2b). In terms of fluorescence, the PQC molecules exhibited similar emission spectra to PA in methanol (FIG. 2c).

### Construction and Characterization of PQC NFs

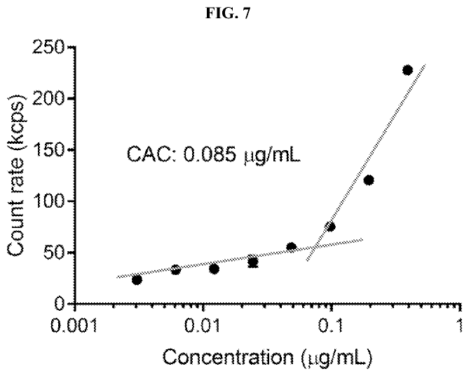

The self-assembling PQC NFs were prepared via a nanoprecipitation method, in which the PQC solution in ethanol was added into water dropwise, followed by the evaporation of ethanol under reduced pressure. By using transmission electron microscopy (TEM), we found that PQC molecules self-assembled into the uniform nanofiber scaffold networks with average diameters of 14.9±2.5 nm and a surface charge of 42.3±1.3 mV (FIG. 2d). The positive surface charge comes from the cationic quinolinium, indicating that quinolinium moieties spread over the surface of PQC NFs. The critical aggregation concentration (CAC) of PQC NFs was measured to be 0.085 μg mL⁻¹ (FIG. 7). The ultrafiltration method was then employed to identify the formation of PQC NFs (FIG. 2e). The majority of PQC NFs were retained in the centrifugal filter (10 kDa), showing a dark green color and strong fluorescence, while the colorless filtrate with low fluorescence indicated only a trace amount of PQC molecules.

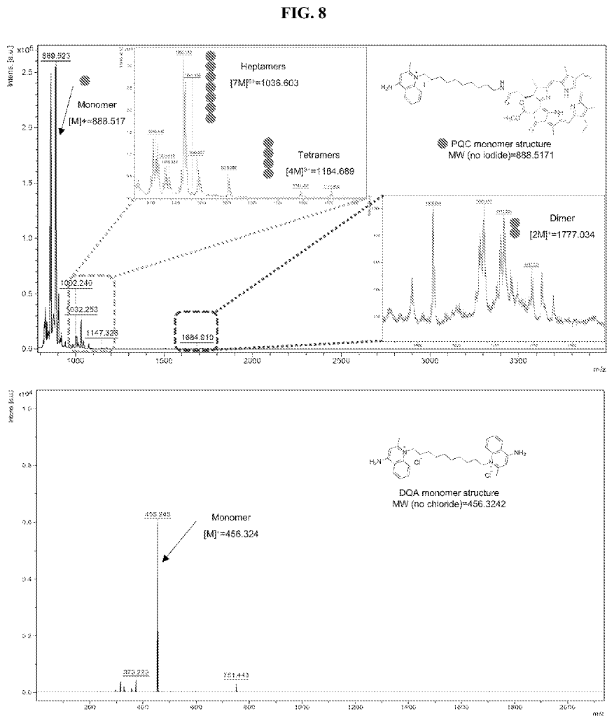

The morphological structure of amphiphilic self-assembled aggregates depends on the relative size of the hydrophobic and hydrophilic moieties. In the PQC molecule, there are a large hydrophobic PA group (mw=590) and a small hydrophilic quinolinium group (mw=158). This high hydrophobicity-to-hydrophilicity ratio determines the aggregation of PQC molecules into nanofibers. The formation of aggregates also benefits from the strong π-π stacking interactions among PA moieties of PQC molecules. MS was employed to explore the structure of PQC aggregates in aqueous conditions, which is a powerful tool to investigate the assembly of small molecules. The PQC monomers (m/z 888.517), dimers (m/z 1777.034), tetramers (m/z/4 1184.689) and heptamers (m/z/6 1036.603) were observed from MS (FIG. 8), suggesting that the PQC molecule is the building block that aggregates into nanofibers. DQA, the control, which has been demonstrated to self-assemble into liposome-like vesicles (DQAsomes), was also included in this study. As the interactions of hydrophobic chains are very weak, DQA did not show any signals of multimer in the MS spectra (FIG. 8).

Without a bio-cleavable chemical bond in its chemical structure, the PQC monomer is not a prodrug form of an existing photosensitizer, but a new chemical entity that possesses an excellent self-assembling property. Therefore, compared to the majority of traditional nanoformulations that are prepared by physical loading or prodrug self-assembly of existing drugs, the new-chemical-entity-assembled PQC NFs represent a structure innovation in the perspective of new drug discovery. Additionally, the one-component PQC NFs have a 100% drug loading efficiency and show enormous advantages to break through the drug-loading and scale-up production limitations of the conventional drug delivery systems.

### Optical Property and ROS Production in Solutions

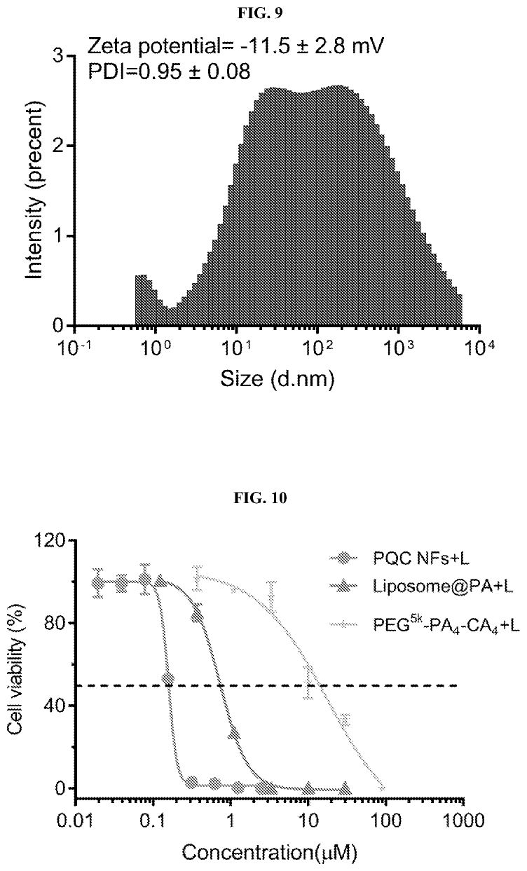

It is known that the PA fluorophore has an aggregation caused quenching (ACQ) effect on its fluorescence emission. Upon the self-assembly, the aggregated PA moieties localized inside the PQC NFs and showed a very weak fluorescence emission (FIG. 2f). After dissociation by sodium dodecyl sulfate (SDS), their fluorescence was recovered. The aggregation-dissociation-based fluorescence “off-on” behavior was also observed with near-infrared (NIR) fluorescence imaging (FIG. 2g). The PQC NFs in PBS showed low fluorescence due to the ACQ effect, and when dissociated in PBS/SDS, they exhibited a concentration-dependent fluorescence enhancement as expected. The hydrophobic PA compound also showed a similar ACQ behavior as PQC NFs, indicating PA was aggregated as well in PBS. However, because the carboxylic acid group is not an excellent hydrophilic moiety, the aggregates of PA displayed a scattered size distribution from nanometers to micrometers and a negative surface charge of −11.5±1.3 mV (FIG. 9). As the pioneering theranostic agents, porphyrin derivatives can be used for both photodynamic therapy and NIR fluorescence imaging. This “off-on” fluorescent property can be used to track the permeability and persistence of PQC nanofibers in tumor sites specifically.

When absorbing a specific wavelength of light, photosensitizers can convert oxygen into ¹O₂, which consequently causes an increase of ROS and is a critical anticancer mechanism of PDT. Moreover, for a photosensitization group, the singlet oxygen quantum yield is an intrinsic property, and modifying the photosensitizer by conjugation with other moieties may cause the decrease of singlet oxygen production efficacy in solution. Singlet oxygen sensor green (SOSG) was used as a probe to determine the ¹O₂ production induced by PA and PQC NFs in solutions. As shown in FIG. 2h, PQC NFs produced a similar amount of ¹O₂ with PA at the same concentration, which indicates that chemical conjugation did not impede the ability of ¹O₂ production. In addition, both PQC NFs and PA produced limited ¹O₂ in the aggregation forms (in PBS), while their dissociated forms (in PBS/SDS) showed an increased capacity of ¹O₂ production (FIG. 2h), indicating the ¹O₂ production can be specifically activated by their free molecules, rather than their aggregates. These results are consistent with our aforementioned findings of the fluorescence “off-on” behavior of PQC NFs, which allows for specific photodynamic reactions for use as highly selective cancer therapies.

### In Vitro Anticancer Activity

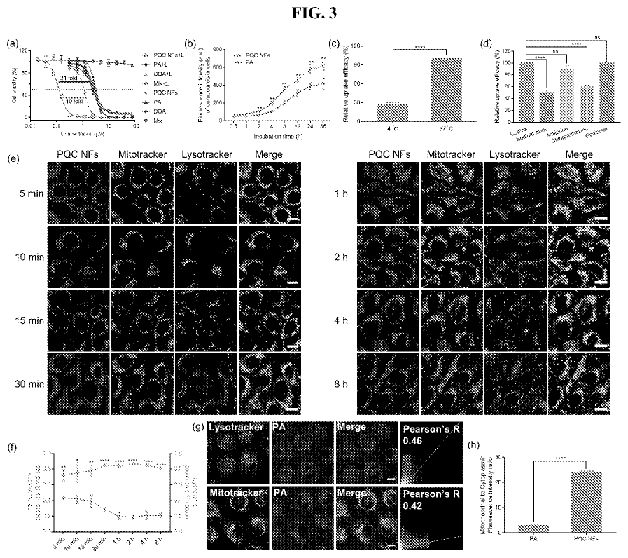

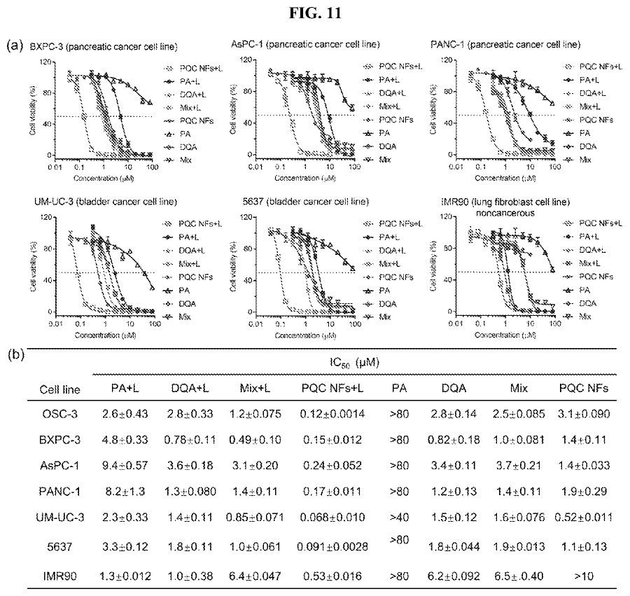

After characterizing the properties and functions of PQC NFs in solutions, we moved to evaluate the in vitro anticancer effects in cells. The OSC-3 cell line, a type of superficial oral squamous cell carcinoma, was chosen firstly because PDT is ideally suited to this cancer type and some related therapies have already been approved by FDA for use in the clinic. The viability of OSC-3 cells was measure using the CellTiter-Glo cell viability assay (FIG. 3a). In the absence of light, PA did not show cytotoxicity even at 100 uM, while PQC NFs showed similar cytotoxicity with DQA and the mixed treatment (IC₅₀=3 μM). When cells were exposed to light treatment, PQC NFs showed a dramatically increased anticancer effect against OSC-3 cells (IC₅₀=0.12 μM), which is 10-fold and 21-fold more potent than that of the mixed treatment (IC₅₀=1.21 μM) or PA (IC₅₀=2.57 μM), respectively. Remarkably, the control groups of DQA, PA, and their mixture did not show obvious anticancer effects at the concentration range from 0.3 μM to 1 μM, while PQC NFs eliminated all the OSC-3 cancer cells at those concentrations. Moreover, PQC NFs were superior to other nanoformulations of PA, such as PA-loaded liposomes (liposomes@PA) and PA-conjugated polymers (PEG^{5k}-PA₄-CA₄) (FIG. 10). These cytotoxicity findings strongly support our hypothesis that delivering photosensitizers into mitochondria can greatly improve their therapeutic effects. In the extended cell viability assays (FIG. 11), it was also found that the light-treated PQC NFs had 32 to 48 times higher potency than light-treated PA in inhibiting pancreatic cancer cells (BXPC-3, AsPC-1, and PANC-1) and bladder cancer cells (UM-UC-3 and 5637), implying their considerable potential to be applied to other cancer types. Compared to these cancer cells, the noncancerous IMR90 cells were more tolerated to PQC NFs in both the absence and presence of light irradiation, which suggests that PQC NFs have relatively low toxicity to the noncancerous cell line (FIG. 11).

### Cellular Uptake and Endocytosis

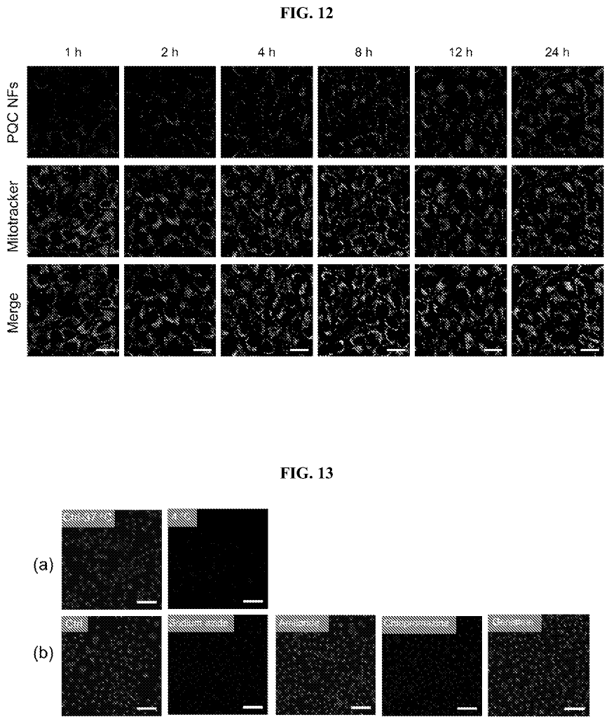

To illuminate the intracellular characteristics of PQC NFs, a cellular uptake study was conducted (FIG. 3b and FIG. 12). PQC NFs showed a time-dependent accumulation in OSC-3 cells and achieved a significantly improved profile of cellular uptake than free PA. This is attributable to the effective self-assembling properties of PQC NFs, including their homogeneous nanosize characteristics and positive surface charge. To interrogate the endocytosis mechanism of PQC NFs, the entry of PQC NFs into cells was measured when incubation was performed at low temperature. The results show the decrease in temperature inhibited cellular uptake of PQC NFs, which suggests the endocytosis PQC NFs is energy-dependent (FIG. 3c and FIG. 13). Four types of endocytosis inhibitors were then utilized to reveal the specific endocytic pathway of PQC NFs: sodium azide, chlorpromazine, genistein, and amiloride. Among these, both sodium azide and chlorpromazine decreased the cellular uptake of PQC NFs, which implicated that PQC NFs entered cells mainly through clathrin-mediated endocytosis, rather than caveolae-mediated endocytosis (genistein) or micropinocytosis (amiloride) (FIG. 3d and FIG. 13).

### Mitochondrial Targeting

We subsequently investigated the subcellular localization of PQC NFs by using confocal microscopy. OSC-3 cells were incubated with PQC NFs for different time points (from 5 min to 8 h), followed by staining cells with MitoTracker and LysoTracker before imaging. As depicted in FIG. 3e, PQC NFs (red) and mitochondria (green) colocalized quickly (within 5 min) as indicated by the yellow color on the merged overlay. The calculated Pearson correlation coefficients in FIG. 3f indicate a high degree of colocalization between PQC NFs and mitochondria at 5 min (Pearson's R, 0.72), and the colocalization reached the maximum in 2 h (Pearson's R, 0.87). However, the colocalization with lysosomes was only moderate at 5 min (Pearson's R, 0.43) and decreased in a time-dependent manner. These results demonstrate that PQC NFs can quickly enter the mitochondria of living cells and remain there for hours. In contrast, the parental PA molecules were diffused into the cytoplasm and exhibited a low-level colocalization with mitochondria or lysosomes, indicating an obvious difference with the mitochondria-targeted subcellular distribution of PQC NFs (FIG. 3g). We also isolated the mitochondria and cytoplasm of OSC-3 cells following a 24 h pretreatment with PQC NFs or PA to quantify the differences in their localization. The PQC concentration in mitochondria was 24-fold higher than that in the cytoplasm, while PA molecules showed a similar concentration in both mitochondria and cytoplasm, confirming that PQC NFs are specifically targeted to mitochondria (FIG. 3h).

### Intracellular Dissociation

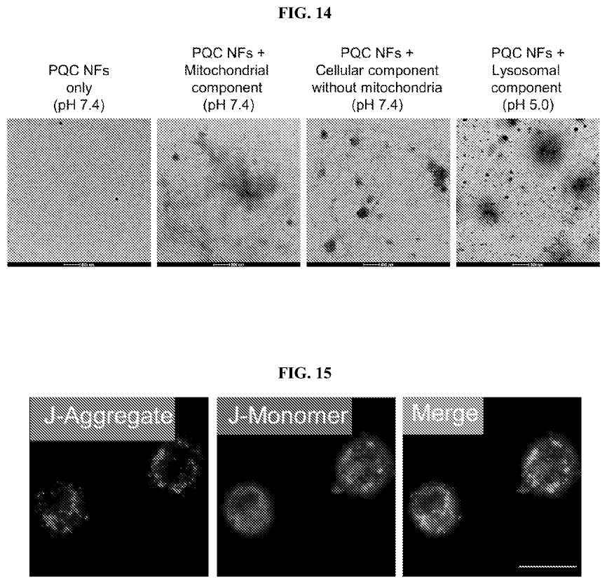

We next investigated the intracellular dissociation behavior of PQC NFs, in which PQC NFs (100 μM) were incubated with the freshly isolated mitochondria (0.1 mg mL⁻¹), lysosomes (0.1 mg mL⁻¹), and other cellular components (0.1 mg mL⁻¹) for 24 h and were then observed by TEM (FIG. 14). It was found that PQC NFs stayed in the aggregation state upon incubation with the lysosomal or cellular component that did not contain mitochondria, but were dissociated in presence of the mitochondria component. This is because the mitochondria inner membrane with strongly negative potential can bind to the delocalized cationic quinolinium moiety on the surface of PQC NFs, which drives the dissociation of NFs into free molecules. Based on these findings, we can conclude that PQC NFs maintained their nanostructures in the process of cell endocytosis and intracellular transport toward mitochondria, and then were dissociated into free molecules in mitochondria. Since the free form of PQC NFs can produce more ROS than the aggregation form (FIG. 2h), the dissociation of PQC NFs that selectively occurs in mitochondria can be conducive to improve the efficiency of PDT.

### Intracellular ROS Production

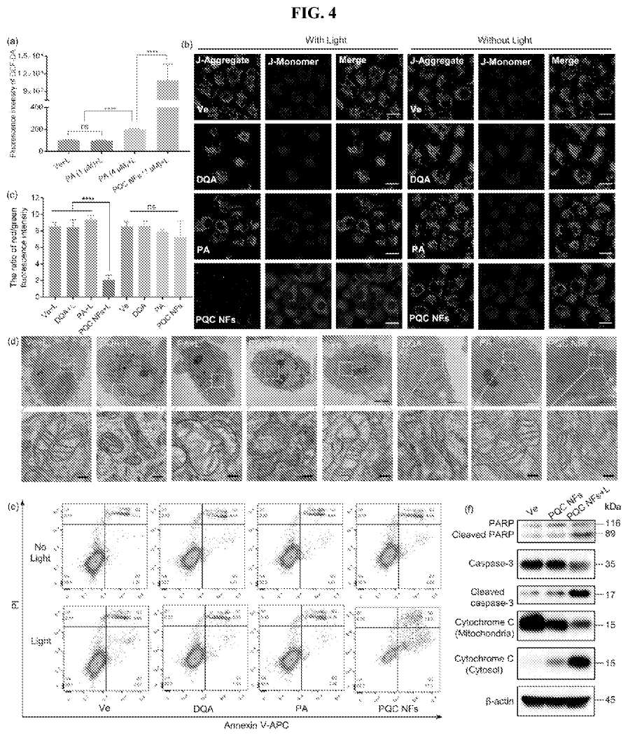

As ROS overproduction is the key anticancer mechanism of PDT, we measured the ROS levels in OSC-3 cancer cells treated with PQC NFs by using 2′,7′-dichlorofluorescein diacetate (DCF-DA) as a fluorescence sensor. After exposure to light, PQC NFs produced approximately 110-fold higher amounts of ROS than the same concentration of free PA (FIG. 4a). Because the PQC NFs and PA have a similar ¹O₂ quantum yield, and the intracellular concentration of PQC NFs is only 1.4 times as that of PA (FIG. 2h and FIG. 3b), this significant enhancement of intracellular ROS production should be attributable to their specific mitochondria targeting property of PQC NFs. As mitochondria are an important source of ROS in most mammalian cells, mitochondria-targeted PQC NFs potentially caused ROS burst in situ through mitochondrial imbalance.

### Mitochondrial Disruption

To get direct insights in PQC NF-mediated mitochondrial damage, the mitochondrial membrane potential was determined using indicator dye JC-1. JC-1 forms J-aggregates in cells with high mitochondrial membrane potential and emits red fluorescence, while it remains monomeric in cells with low mitochondrial membrane potential and emits green fluorescence. Representative results from PQC NFs treatment of OSC-3 cells are displayed in FIG. 4b. No differences were observed among the groups without light treatment, indicating the relatively low dark toxicity of PQC NFs toward mitochondria. Remarkably, the simultaneous treatment of cells with PQC NFs (1 μM) and light caused a decline of red fluorescence signals and the rise of green fluorescence signals, which indicates a severe loss of mitochondrial membrane potential. In contrast, the control treatments with PA or DQA upon light illumination were not effective enough to cause a signal change at the same condition of 1 μM. Treatment with a higher concentration of PA (4 μM), the positive control, was milder when compared to 1 μM PQC NFs as those treated cells still exhibited red fluorescence (FIG. 15). To confirm the visual results, the calculated red-to-green fluorescence ratio of cells in each treatment further showed the potent ability of PQC NFs to induce mitochondria depolarization (FIG. 4c).

We next used TEM to investigate the morphological changes in mitochondria induced by PQC NFs treatment. As shown in FIG. 4d, the vehicle or PA-treated cells without light that showed healthy mitochondria with typical tubular cristae and crista junctions in the absence of light, while DQA-treated cells displayed morphologically changed mitochondria with and without light treatment. This change is likely because the accumulation of DQA in mitochondria interferes with the functioning of membrane proteins and inhibits the mitochondrial respiratory chain. PQC NFs without light treatment were found to cause similar alterations in mitochondria to those observed with DQA, showing that PQC NFs can affect mitochondrial activity. Cells treated with both PQC NFs and light treatment manifested seriously damaged mitochondria, which were indicated by severe cristae disruptions and abnormal vacuoles. In contrast, treatments with PA and light only caused some mitochondrial swelling and the normal mitochondrial microstructures remained intact. Altogether, these findings suggested that PA has very limited effects on mitochondrial photodamage compared to PQC NFs. Due to their mitochondria targeting properties, the light-treated PQC NFs predominantly cause a rapid rise of ROS levels within mitochondria and thus caused mitochondrial imbalance and damage in situ. As mitochondria are the indispensable powerhouses of cells, PQC NF-induced mitochondrial damage can be a death blow for cancer cells.

### Apoptosis Induction

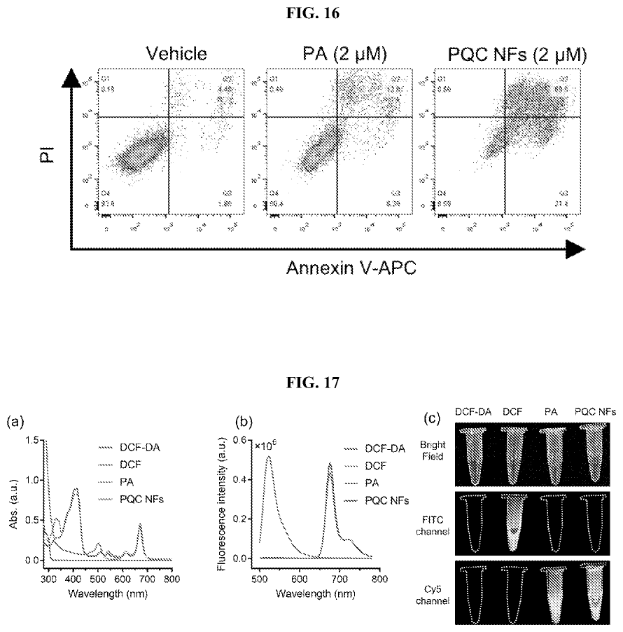

Decreased mitochondrial membrane potential and subsequent mitochondrial damage are landmark events in the early stages of apoptosis. Hence, we explored the downstream apoptotic effects of mitochondrial damage upon treatment with PQC NFs. The total apoptotic cell population was examined by using double staining with annexin V-APC and propidium iodide (PI). PQC NFs with light generated the highest apoptotic populations among all groups even at a low treated concentration (0.2 μM), while PQC NFs without light did not show proapoptotic effects, suggesting the high phototoxicity and low dark toxicity of PQC NFs (FIG. 4e). It should be highlighted that the light-treated control photosensitizer PA failed to induce apoptosis under the same conditions, which can be attributed to its relatively low concentration. When the concentration was increased to 2 μM, the Annexin V positive population of PQC NFs group reached 90.9%, while that of PA was only 19.3% (FIG. 16). These results fully reveal the potent pro-apoptotic effect of PQC NFs.

To identify the apoptotic pathways activated by PQC NF-mediated phototherapy, we measured the changes in traditional apoptosis makers through western blot assays. During the process of apoptosis, the cytochrome c sequestered in the intermembrane space of mitochondria can be released into the cytosol, which leads to the activation of the cytochrome c-dependent caspase cascade. The cytochrome c levels in cells treated with PQC NFs and light were dramatically decreased within mitochondria and increased in the cytoplasm, indicating that PQC NF-induced photodamage could release cytochrome c from mitochondria into the cytosol (FIG. 4f). Meanwhile, the downstream apoptotic proteins, including the caspase-3 and its substrate poly (ADP-ribose) polymerase (PARP), were also found cleaved upon the treatment with PQC NFs and light (FIG. 4f). Taken together, these results validate that PQC NF-triggered PDT can potentially induce mitochondrial disruption and ultimately cause the apoptosis of cancer cells through the cytochrome c-dependent caspase cascade.

### Long-Term Retention in Tumor

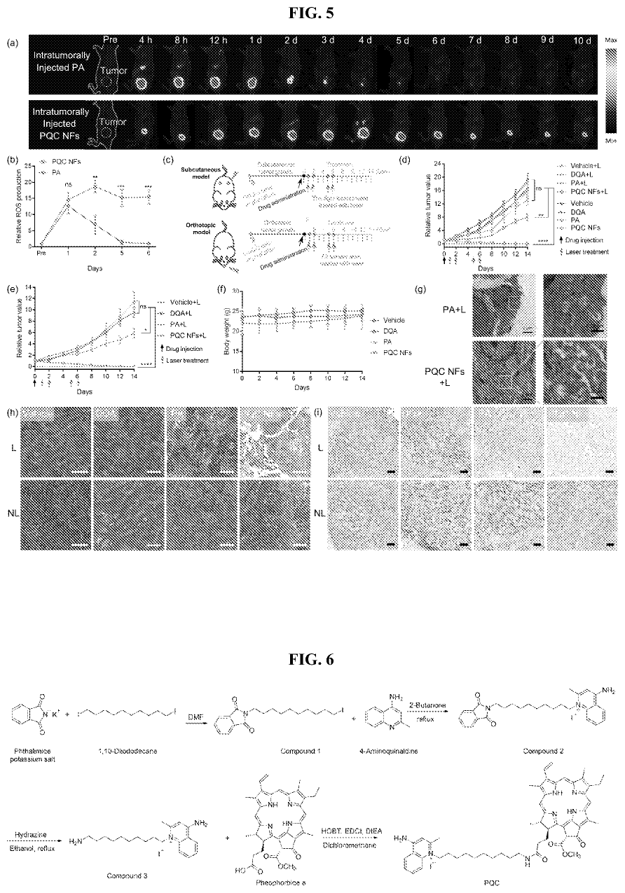

As small molecules, the traditional photosensitizers suffer the rapid clearance from tumors. Rapid clearance can result in the low therapeutic concentrations and poor retention of photosensitizers in tumor sites, leading to insufficient therapeutic efficacy and frequent intakes of medicines. Currently, preparing the nanoformulations of a photosensitizer is an effective strategy to overcome these shortcomings in PDT. Given their high surface-area-to-volume ratio, the fiber-shaped materials can form strong interactions with biosurface and have enormous potentials to be retained in tumor sites. The oral cancer mouse model was established to verify the corresponding advantages of PQC NFs in tumors because this cancer type is readily accessible to both the illumination with laser, a requirement for effective phototherapy, and the topical (intratumoral) administration of phototherapeutic agents. When PQC NFs were injected intratumorally in nude mice bearing OSC-3 tumors, we found that the PQC NFs exhibited fluorescent signals at the tumor site that persisted for nearly a week longer than free PA (FIG. 5a). The fluorescence of PQC NFs within tumors was maintained at a high level for more than ten days, while that of PA was nearly undetectable at the 4th day post-injection, indicating PQC NFs can be retained in tumors for a significant amount of time.

### Intratumoral ROS Production

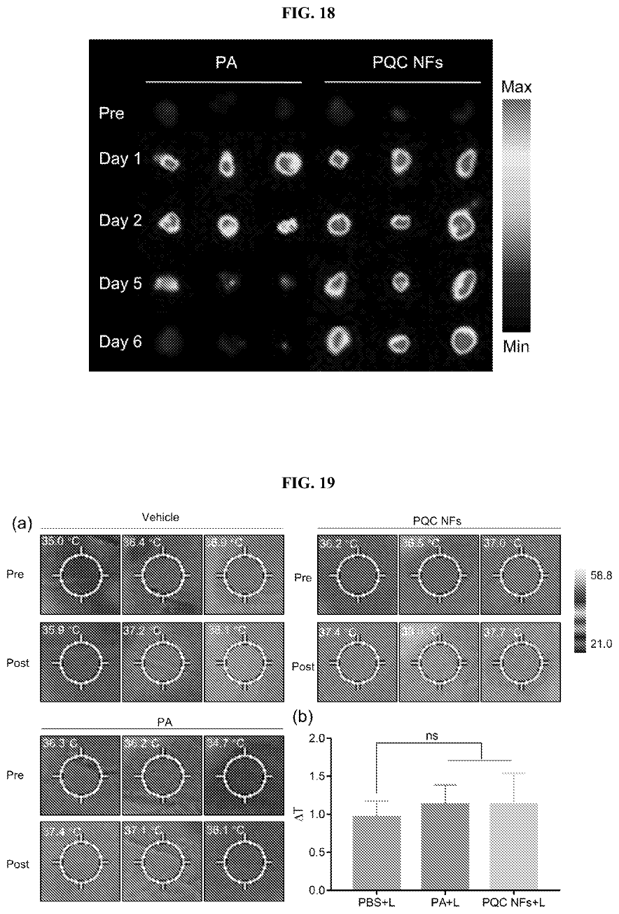

With the advantage of long-term retention, PQC NFs can support multiple light treatments after a single-dose administration. To verify this hypothesis, the light-triggered ROS production in the tumor was monitored at different time points post-injection by using DCF-DA as an indicator. The emission spectra of DCF were not overlapped with that of PQC NFs or PA, indicating that the retained PQC NFs or PA in tumors would not interfere with ROS signals (FIG. 17). ROS levels in tumors treated with PA were high at 24 h post-injection and decreased quickly at later time points, while ROS levels in PQC NF-treated tumors were continuously maintained at a high degree during 6 days (FIG. 5b and FIG. 18). These findings suggest that PQC NFs are very potent photodynamic therapy agents.

### In Vivo Antitumor Effect

To evaluate the in vivo therapeutic effects of PQC NFs, we established a subcutaneous oral cancer model by implanting OSC-3 cells into the flanks of nude mice to evaluate the phototherapeutic and non-phototherapeutic effects of each drug (FIG. 5c). Tumors on the right side were subjected to laser exposure, while the left side tumors were not. All the mice bearing tumors were randomly assigned into four groups: Vehicle, DQA, PA, and PQC NFs, and were treated according to the treatment schedule shown in FIG. 5c. Drug treatment was performed by intratumor injection only once at the beginning of treatments (at Day 0), followed by 4 laser treatments at Day 1, Day 2, Day 5, and Day 6. A low laser power (0.2 W cm⁻², 6 min) was chosen to minimize the interference from photothermal effects because the phototherapy under this condition did not increase the tumor temperature significantly in both PQC NFs and PA groups (FIG. 19). Compared to the vehicle group, the groups without laser (PA, QDA, and PQC NFs) did not show significant antitumor efficacy, suggesting that chemotherapeutic effects of the single-dose drug treatment were very limited (FIG. 5d). The groups of vehicle and DQA with laser also did not show an obvious antitumor effect, indicating that laser treatments were not able to interfere with tumor growth in the absence of photosensitizers. The free photosensitizer PA with laser (PA+L) effectively inhibited tumor growth but did not shrink the tumors. Most interestingly, the laser-treated PQC NFs exhibited the best antitumor efficiency, which achieved a 100% complete cure rate.

The orthotopic oral cancer model was also established by implanting OSC-3 cells into the lips of nude mice (FIG. 5c). In the orthotopic model, all the tumors were involved in phototherapy with mice being divided into the same drug-treated groups (vehicle, DQA, PA, and PQC NFs) as the subcutaneous model and drug treatment being performed by intratumoral injection on Day 0, followed by 4 laser treatments (FIG. 5c). The therapeutic results in the orthotopic model were consistent with those observed in the subcutaneous model, indicating the PQC NFs eliciting a significantly improved phototherapeutic activity over the free photosensitizer PA (FIG. 5e). In addition, as shown in FIG. 5f, all treatments did not cause a loss in body weight of mice.

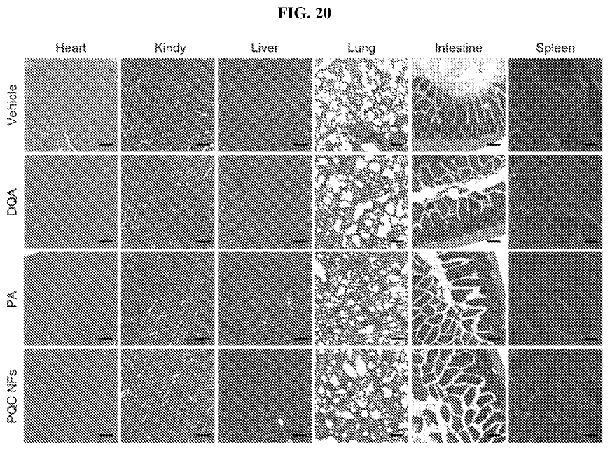

The subcutaneous tumors, which were treated with drugs for 24 h, followed by laser treatment or not, were collected for further investigation of tissue-level changes. From TEM, we observed that the laser-activated PQC NFs induced obvious disruption of mitochondrial microstructures of tumors (FIG. 5g). Hematoxylin and eosin (H&E) staining results showed that tumor cells were predominantly dying or destroyed in tumors treated with PQC NFs+laser, while only a small portion of tumor tissue showed apoptosis/necrosis in the PA with laser group (FIG. 5h). The nuclei were intact and showed bright blue staining in both vehicle and DQA groups, indicating no significant cell death occurred in these groups. Immunohistochemical (IHC) analysis against Ki67 demonstrated that cell proliferation in the laser-treated PQC NFs group was significantly suppressed compared to all other groups (FIG. 5i). In the H&E staining results of normal organs (FIG. 20), there were no obvious changes of their physiological morphology found among the groups, indicating the relatively high safety of PQC NFs. These in vivo therapeutic results fully demonstrated that PQC NFs are promising agents to improve photodynamic cancer therapy.

### Conclusion

In summary, according to the strategies of photosensitizer improvement and the principles of new drug design and molecular self-assembly, we developed a new mitochondria-targeting small-molecule photosensitizer that can self-assemble into nanofibers (PQC NFs). By targeting and disrupting mitochondria, the phototherapy mediated by PQC NFs induced significant apoptosis and exhibited nanomolar cytotoxicity. This targeting approach overcomes insufficient potency of traditional photosensitizers as anticancer therapies. Hence, PQC NFs showed significantly improved in vitro anticancer potency compared to both free and nanoformulated PA. Additionally, as nanoscale materials, PQC NFs also demonstrated long-term retention in tumor sites, solving the challenge of rapid clearance from tumors found in existing small-molecule photosensitizers. With these advantages, PQC NFs achieved a significant antitumor effect in vivo by affording a 100% complete cure rate on both subcutaneous and orthotopic oral cancer models with only a single-dose administration.

In contrast to the conventional nanoformulations that are prepared by physical loading or prodrug self-assembly of existing drugs, PQC NFs are formed by the self-assembly of one-component new chemical entities, which not only represent a structural innovation in the perspective of new drug discovery but also show enormous potentials to break through the drug-loading and scale-up production limitations of the conventional drug delivery systems. Furthermore, PQC NFs also have the unique fiber-shaped nanostructure that is rarely found among the existing small-molecule nanomaterials because the majority of developed nanofibers are based on the peptides with a specific sequence. To the best of our knowledge, PQC NFs represent the first example of single small molecule-assembled nanophotosensitizers, which refer to a transdisciplinary design strategy to advance the phototherapeutic efficiency of traditional photosensitizers from both perspectives of molecule design and nanoformulation.

### Example 3. Conjugates

### Formula of Quinolinium-Drug Conjugates:

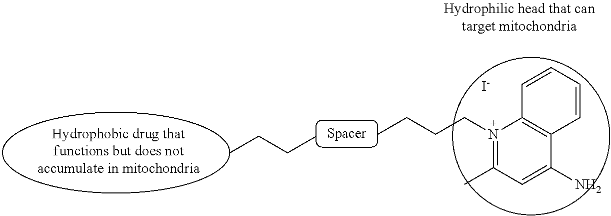

The hydrophobic core are those drugs that can play their roles in mitochondria but have low subcellular mitochondrial localization. The potential selected drugs are pheophorbide a (photosensitizer), lonidamine, botulin, betulinic acid, β-lapachone, and α-tocopheryl succinate, pyrvinium, atovaquone, bedaquiline, antimycin A, oligomycin A, rotenone, piericidin A, Atpenin A5, 3-nitropropionic acid, myxothiazol, stigmatellin, aurovertin-B, and trifluoromethoxy carbonylcyanide phenylhydrazone.

The amphiphilic quinolinium-drug conjugates can acquire excellent nanoscale advantages as they can form nano-aggregates via self-assembly. Representative examples are:
pheophorbide a-quinolinium conjugate: This compound can perform a mitochondrial targeting photodynamic cancer therapy. The photosensitizer part can be replaced by other photosensitizers.

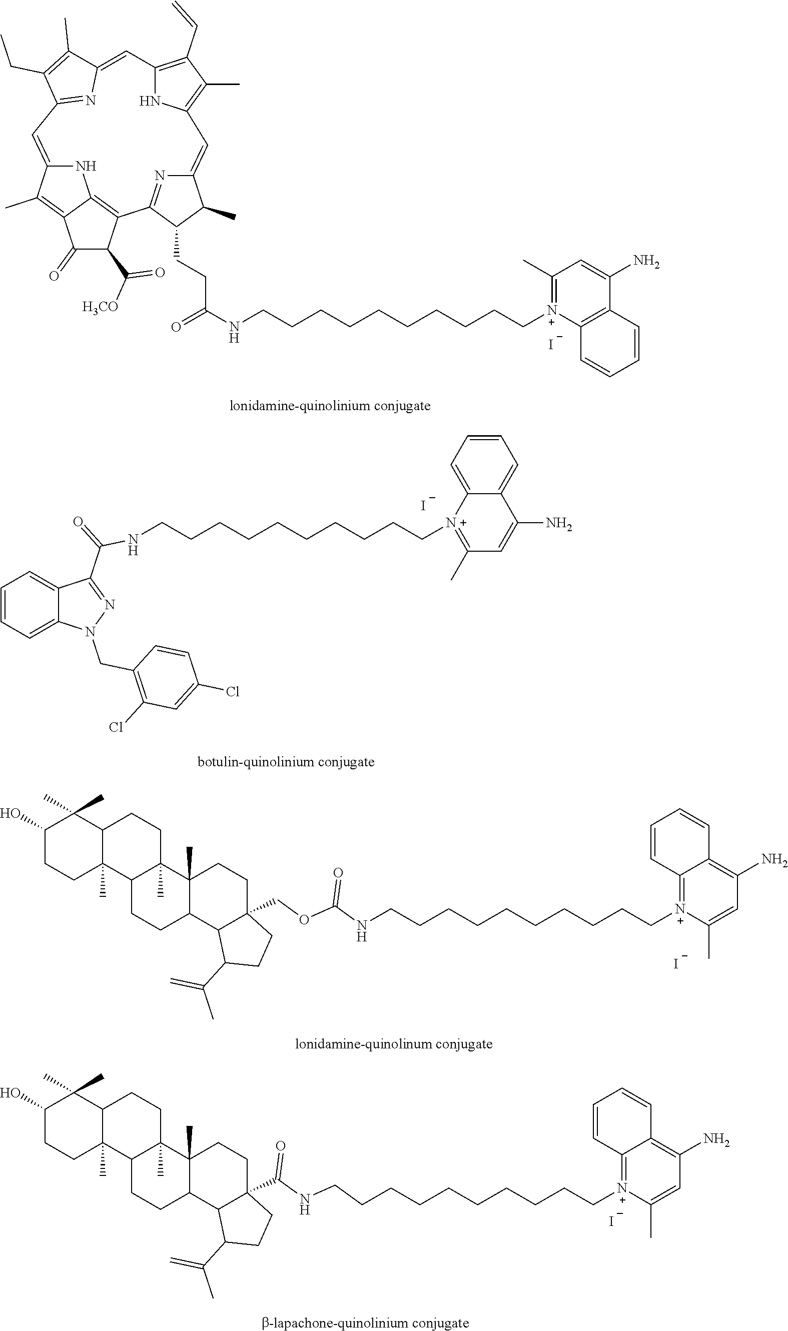

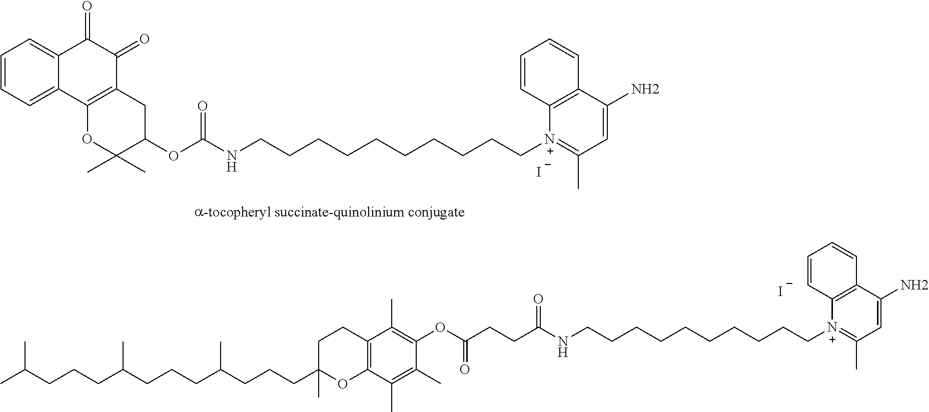 [SMILES: Cc1cc(N)c2ccccc2[n+]1CCCCCCCCCCNC(=O)OC1CC2=C(OC1(C)C)c1ccccc1C(=O)C2=O ; [I-] ; Cc1c(C)c2c(c(C)c1OC(=O)CCC(=O)NCCCCCCCCCC[n+]1c(C)cc(N)c3ccccc31)CCC(C)(CCCC(C)CCCC(C)CCCC(C)C)O2 ; [I-]]

Formula of pH-Responsive Prodrug of Quinolinium-Drug Conjugates:

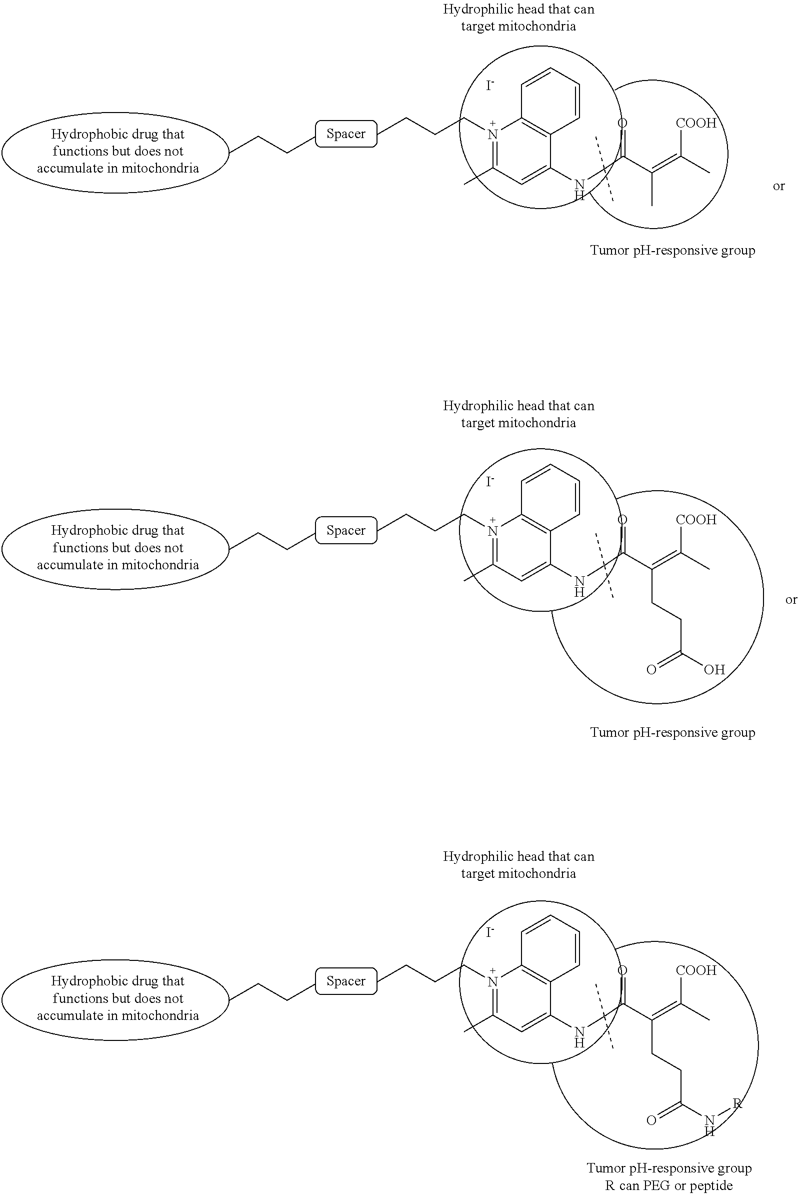

Representative examples are:
Prodrugs of Pheophorbide a-Quinolinium Conjugate

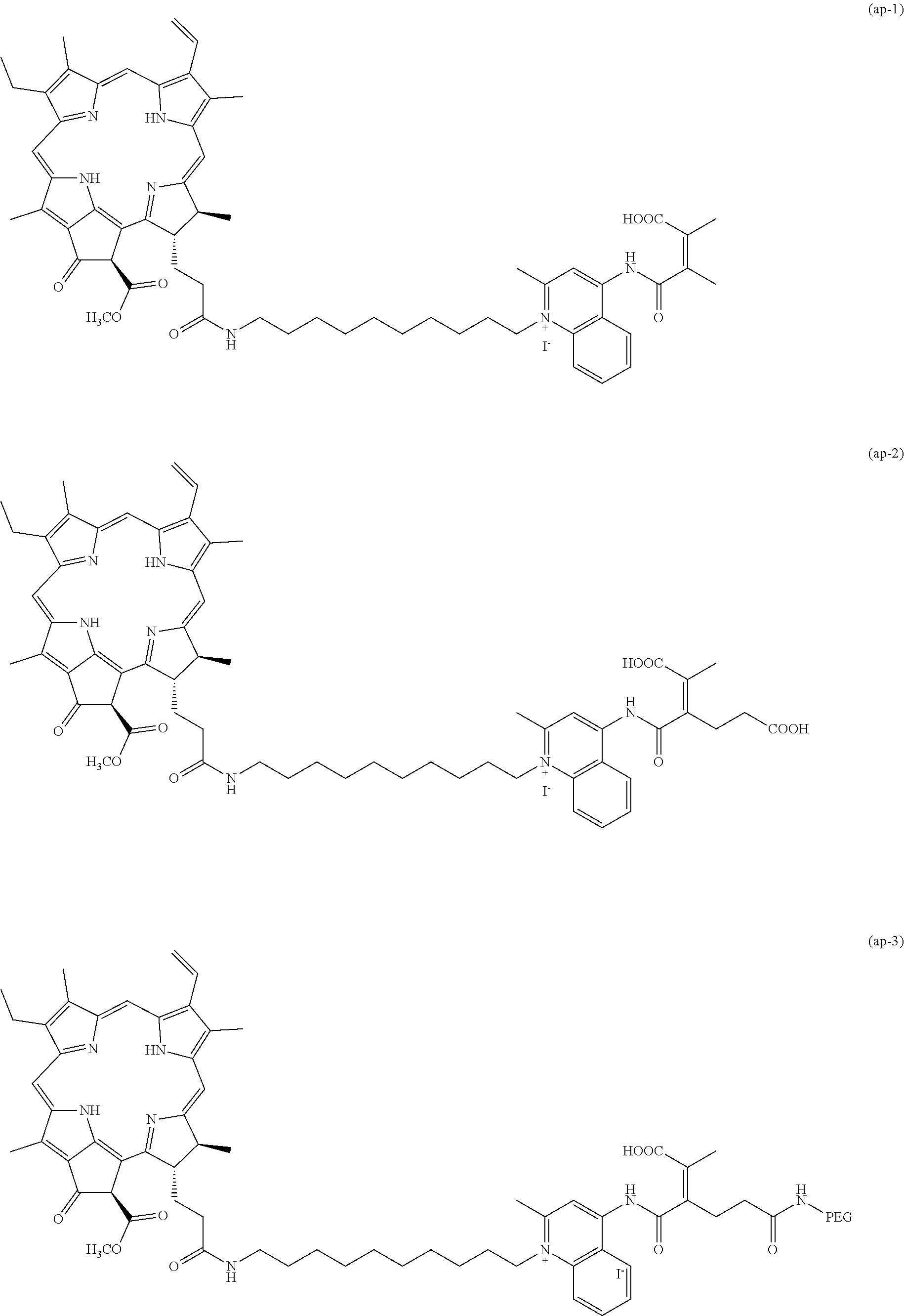

### Prodrug of Lonidamine-Quinolinium Conjugate

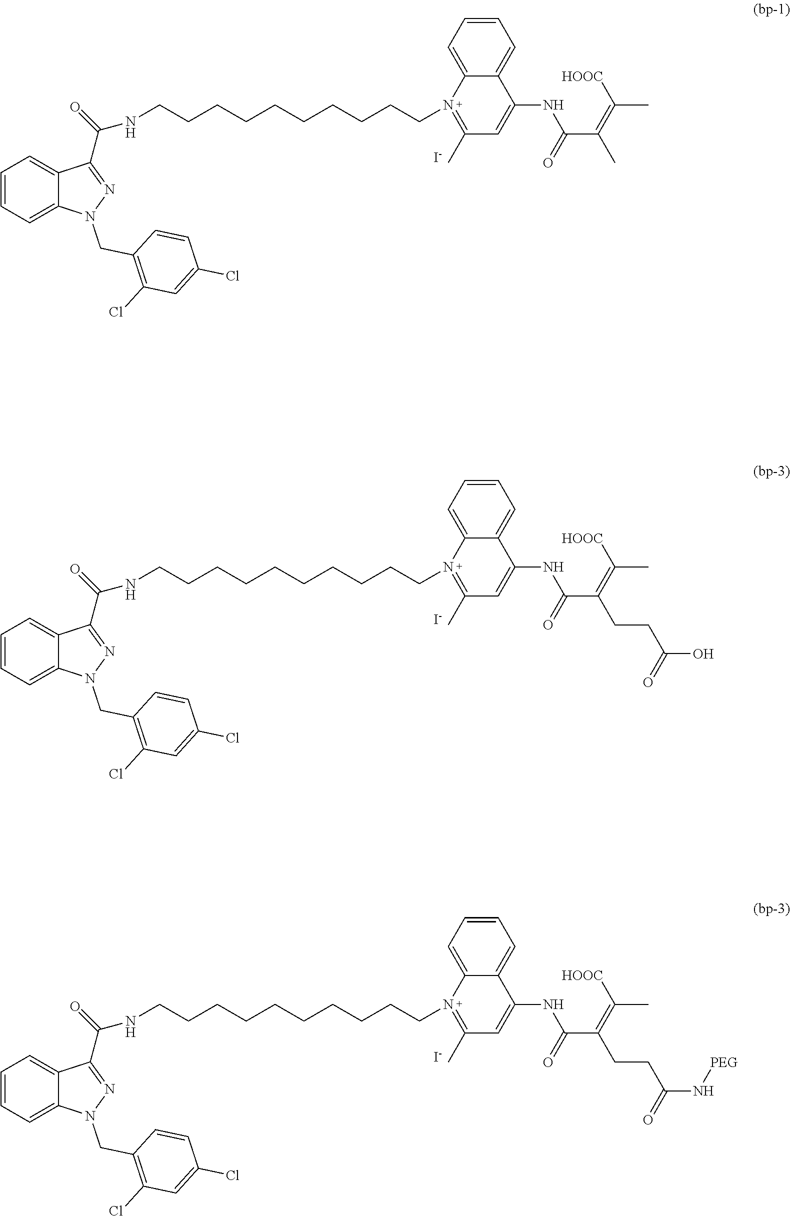

### Prodrug of Botulin-Quinolinium Conjugate

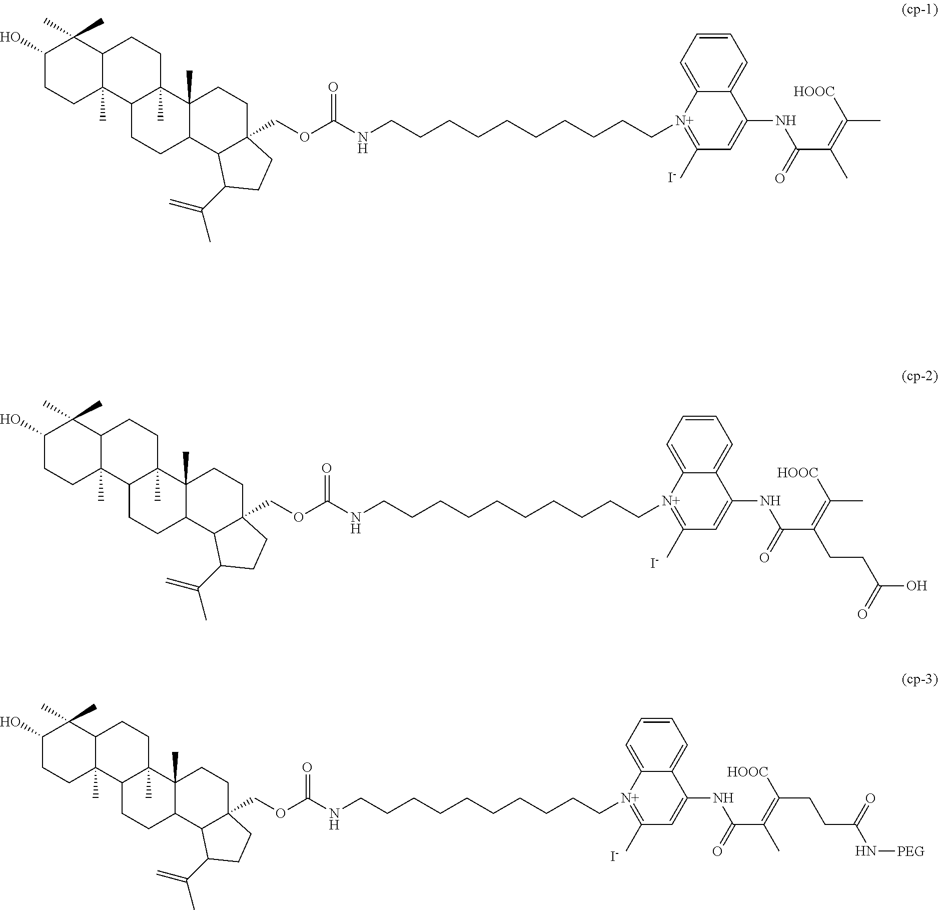

### Prodrug of Lonidamine-Quinolinium Conjugate

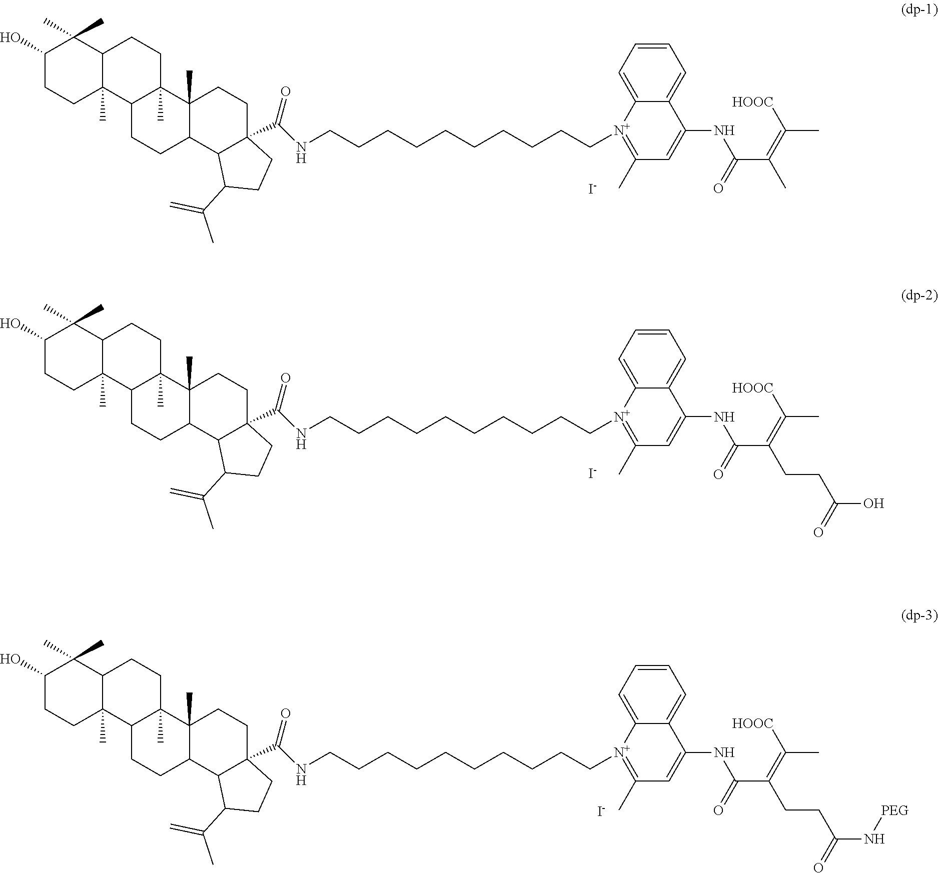

### Prodrug of β-Lapachone-Quinolinium Conjugate

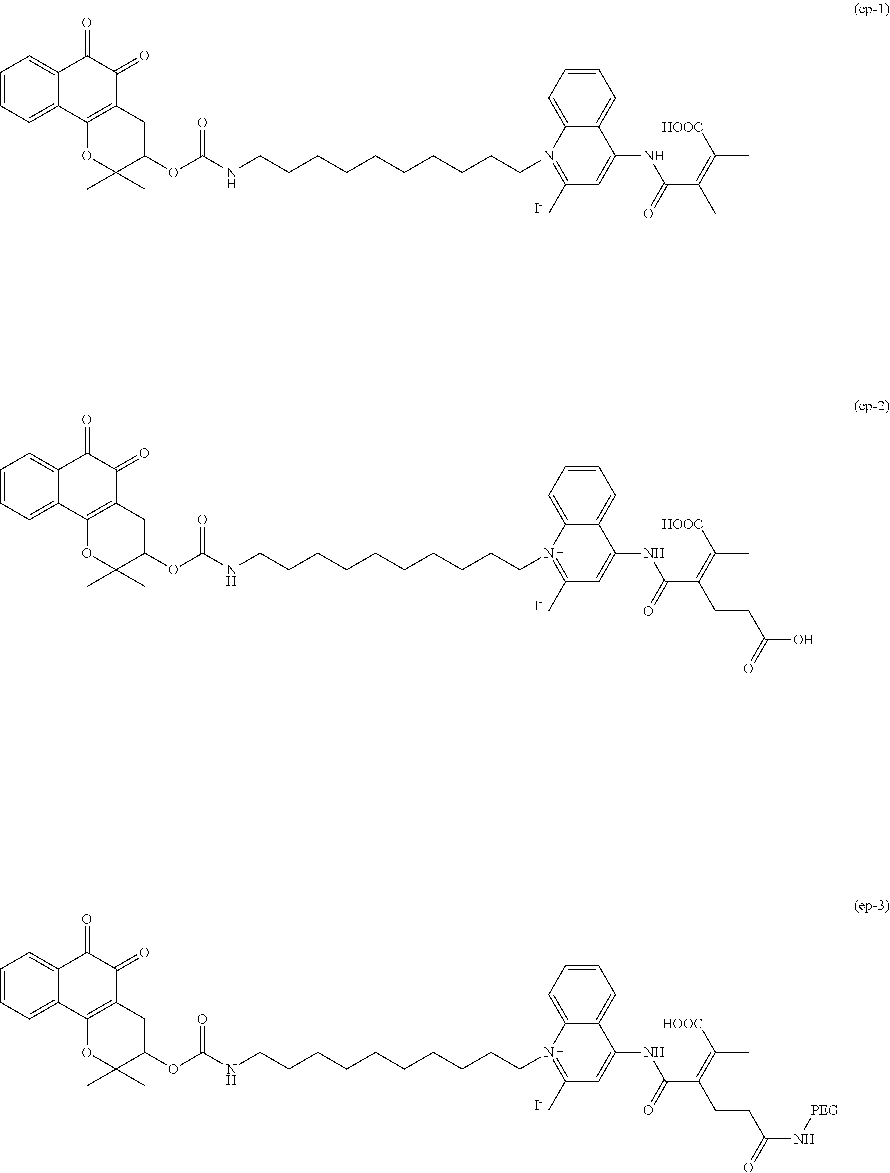

### Prodrug of α-Tocopheryl Succinate-Quinolinium Conjugate

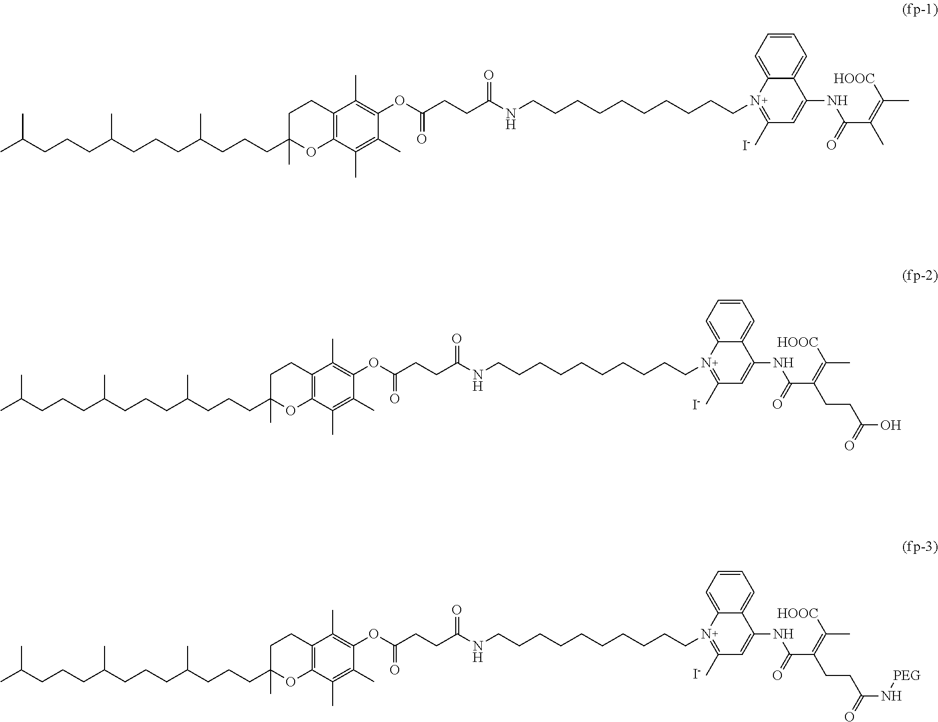

### Example 4. Glioma Imaging and Therapy

### Chemicals

Pheophorbide a was bought from Santa Cruz Biotechnology (TX, USA). L-α-phosphatidylcholine was purchased from Avanti Polar Lipids, Inc (AL, USA). Cholesterol was brought from MP Biomedicals (OH, USA). mPEG-DSPE (MW. 2000) was brought from Laysan Bio, Inc (AL, USA). Organic solvents were purchased from Fisher Scientific (MA, USA).

### Preparation of NPs

LPHNPs and liposome@PA were prepared via a classical thin film hydration method. Briefly, L-α-phosphatidylcholine (Soy PC, 10 mg), cholesterol (2.2 mg), mPEG-DSPE (2.2 mg) and PQC (1 mg) or Pheophorbide a (PA, 1 mg) were dissolved in chloroform and added into a round-bottom flask under stirring. Then, the chloroform was evaporated to form a thin film. 1 mL phosphate buffered saline (PBS) buffer was added to re-hydrate the thin film, followed by low-level shaking overnight.

### Characterization of NPs

UV-vis spectra were obtained with a UV-vis spectrometer (UV-1800, Shimadzu). Fluorescence spectra were collected by a fluorescence spectrometer (RF-6000, Shimadzu) with the excitation wavelength at 412 nm. Size distribution, PDI and zeta potential were measured by dynamic light scattering (DLS) instruments (Malvern, Nano-ZS). Morphology of nano-assemblies was observed by a Talos L120C TEM (FEI) at an accelerating voltage of 80 kV. To calculate the drug load rate, nano-assemblies were cut off by the centrifugal dialysis tube (MWCO: 10 kDa) and the absorbance of filtrate was measured.

### Cell Culture

The original and transfected GL261 (glioblastoma) cells, U118 and U251 were kindly provide by Dr. Kit Lam's lab. U118 and U251 were cultured in the Dulbecco's modified eagle medium (DMEM), containing 10% FBS and 1% penicillin/streptomycin. They were maintained in the Gibco Dulbecco's Modified Eagle Medium: Nutrient Mixture F-12 (DMEM/F-12), containing 1×ITS (Insulin-Transferrin-Selenium) Liquid Media supplement (Sigma, USA), 1×MEM Non-Essential Amino Acids (Thermofisher, USA), 1× GlutaMAX™ (Thermofisher, USA), 10% FBS and 1% penicillin/streptomycin at 37° C. humidified atmosphere with 5% CO₂.

### Cell Viability Assay

GL261, U251 and U118 cells (5×10³ cells/well) was plated in 96-well plates, incubated overnight, and then were treated with different concentrations of agents as indicated. After 24 h treatment, the cells were washed and cultured with fresh medium. For the light treated groups, cells were irradiated for 30 s using a 633-nm LED array (Omnilux new-U, power density: 30 mW/cm²) and further incubated for 24 h in parallel with non-light treated group. Cell viability was quantified using the CellTiter-Glo assay (Promega, USA) and the luminescence intensity was measured by a microplate reader (Molecular Devices, SpectraMax iD5, USA).

### Cell Uptake Assay

GL261 cells (5×10³ cells/well) were seeded in a 96-well plate, incubated overnight for full attachment, and were treated with 1 μM of agents at different time points as indicated. At the end of the experiment, cells were lysed with dimethyl sulfoxide (DMSO) solution. Fluorescence intensity (Ex=412 nm, Em=675 nm) was measured by a microplate reader (Tecan, Switzerland).

### Colocalization Assay

To conduct the subcellular localization assays, GL261 cells were incubated in a cell view dish overnight and treated with LPHNPs or liposome@PA (0.5 μM) for several hours (from 1 h to 24 h), followed by staining with MitoTracker Green (Cell Signaling Technology, USA) for 1 h. Cells were visualized using a confocal laser scanning microscopy (CLSM) (Carl Zeiss, Germany). Signals of LPHNPs or liposome@PA were observed under the Cy5 channel, and MitoTracker were observed under the Alexa Fluor 488 channel. The corresponding Pearson's correlation coefficient was calculated by Fiji.

### Mitochondrial Membrane Potential Analysis

JC-1 dye (Thermo Fisher Scientific, USA) was used as an indicator of mitochondrial membrane potential. Briefly, Cells (2×10⁴ cells/well) were treated as indicated for 24 h, washed and cultured with fresh medium. For light treated group, cells were irradiated for 30 s using a 633-nm LED array (30 mW/cm²) and incubated for 2 h. Then 0.5 μg/mL JC-1 was added for another 30 min incubation. Images were captured by CLSM. The ratio of red/green fluorescence intensity was calculated by Fiji.

### Electron Microscopy

GL261 cells seeded at 2×10⁴ cells/well in 8-well slide plates (Thermo Fisher, USA), were incubated overnight, treated as indicated for 24 h and then were washed with PBS. Then cells were incubated with fresh medium and were treated with or without light for 30 s. After another 2 h incubation, cell fixed with the 0.1 M cacodylate buffer containing 2.5% glutaraldehyde plus 2% paraformaldehyde, and transferred to the carbon square mesh, followed by observation using the Talos L120C TEM (Thermo Fisher, USA).

### Ros/MitoROS Production Assay in Cellular Level

GL261 cells (5.0×10⁵ cells/well) were seeded in 6-well plates, cultured for 24 h. The cells were treated with 1 μM LPHNPs or liposome@PA NPs for 24 h. After washing with PBS, the cells were incubated with DCF-DA (10 μM) or 1× MitoROS™ 580 (AAT Bioquest, Inc., USA) probe for another 30 min, followed by light treatment (633-nm LED array, 30 Mw/cm²) for 30 s. Cells were incubated for 30 min, and then fluorescence intensity was measured by a microplate reader (Tecan, Switzerland). For the confocal images, cells were observed immediately by CLSM after staining.

### Animal Model

All animal experiments were carried out in accordance with guidelines and animal protocol approved by the ethics committee of University of California, Davis. Female C57BL/6 mice (6 weeks old) were purchased from Harlan (Livermore, CA, USA) for orthotopic model establishment. 2 μL of GL261 cells (5×10⁵ cells) were injected into the right striatum of the mouse. Animals received post-surgery for pain management for 3 days.

### In Vivo/Ex Vivo Fluorescence Imaging

C57BL/6 mice bearing orthotopic GL261 tumors were subjected to tail vein injection of LPHNPs (10 mg/kg). Mice were injected with D-luciferin (150 μL of 20 mg/ml) and imaged using Lago X (Spectral Instruments Imaging, USA) at designated time points. After 24 h of injection, mice were sacrificed, and their organs including the brain with tumor were harvested for ex vivo imaging. The whole brain containing tumor was immersed with optimum cutting temperature (O.C.T.) compound and frozen in −80° C., and then cut into 10 μM thick cryo-sections for fluorescence imaging.

### In Vivo Antitumor Studies

One week after tumor implantation, C57BL/6 mice bearing orthotopic GL261 tumors were randomly divided into three groups: PBS, LPHNPs and liposome@PA. LPHNPs and liposome@PA (10 mg/kg) were injected via tail vein for one dose on day 0. For the light treated groups, the right side of the brain was irradiated with a NIR laser system (Shanghai Xilong Optoelectronics Technology, China) at 680 nm at 0.2 W/cm² for 3 min after 24 h and 48 h of drug administration. Tumor growth was monitored over time by recording bioluminescence signals (mouse was injected with 150 μL of 20 mg/mL D-luciferin) using Lago X (Spectral Instruments Imaging, USA). At the end of experiment, mice were sacrificed, and the tumors were harvested for histopathology analysis.

### Statistical Analysis

The experimental data were statistically analyzed using the GraphPad Prism 7.0. Data were reported as the mean±standard deviations (SD). Data statistics were analyzed by calculating the t-test between two groups. ns not significant; *p<0.05; **p<0.01; ***p<0.001; ****p<0.0001

### Preparation and Characterization of the LPHNPs.

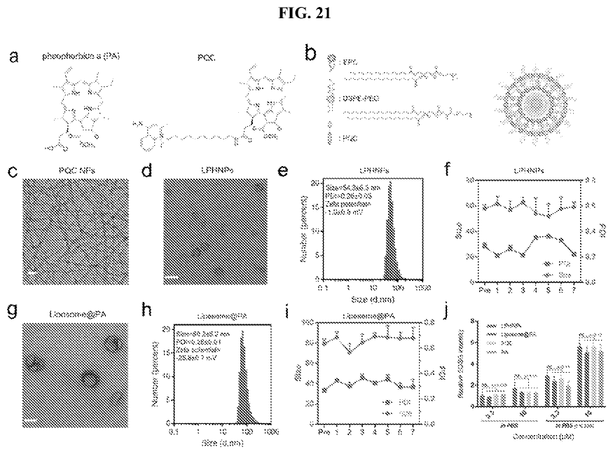

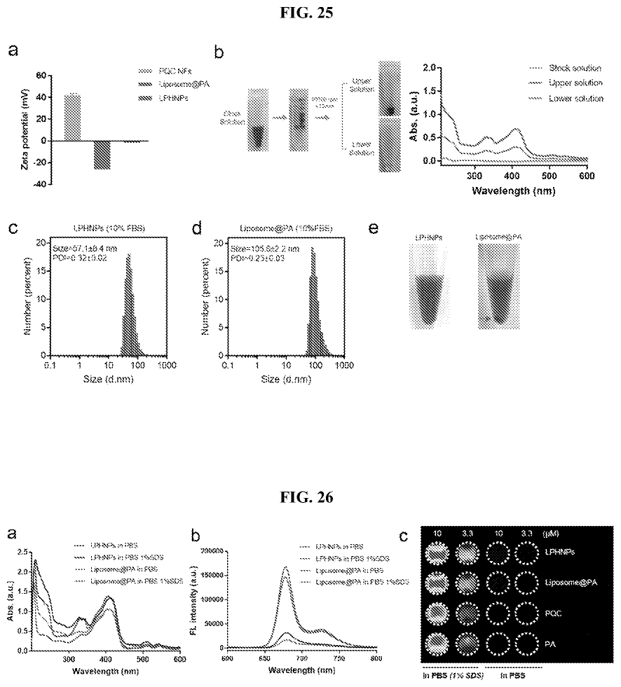

As we presented previously, PQC molecules, the active pharmaceutical ingredient that target mitochondria tend to form nanofibrils (PQC NFs), which is conducive to the retention in tumor of agents but not to their blood circulation (FIG. 21a, FIG. 21c). To adjust and optimize the micromorphology of PQC nano-assemblies, we introduced the amphiphilic lipid for co-assembly. The typical thin-film hydration method was utilized to prepare LPHNPs (FIG. 21b). Transmission electron microscopy (TEM) studies showed that LPHNPs have a uniform and typical core-shell vesicular microstructure (FIG. 21d). This result is consistent with the dynamic light scattering (DLS) measurements, by which the hydrodynamic size, polydispersity index (PDI), and surface charge of LPHNPs were determined to be ˜54.3 nm, ˜0.26, and −1.9 mV, respectively (FIG. 21e). To better elucidate the superiority of the hybrid nanoparticle, we also made a conventional nanoformulation (liposome@PA) that physically loads the hydrophobic PA in liposomes. Under the same drug loading content with LPHNPs, liposome@PA displayed irregular microstructures with several cavities and uneven thickness of films despite the similar DLS result to that of LPHNPs (FIG. 21g-FIG. 21h). This is likely due to the high hydrophobicity and strong π-π stacking of PA as well as the resulting varied aggregation and distribution in the hydrophobic bilayer. What should be emphasized is that compared with the high-positively charged PQC NFs (˜40 mV) and high-negatively charged liposome@PA, LPHNPs displayed the neutralized surface charges (FIG. 25a), which reflects that the interaction between the lipid and PQC molecules on the surface of nano-assemblies (FIG. 21b). Co-assembly of lipids with PQC qualified a high drug loading capacity (up to 55%) with an excellent encapsulation efficiency (92%) (FIG. 25b). In addition, LPHNPs were stable in the long-term (one week) storage or in presence of 10% serum (FIG. 21f, FIG. 25c). Liposome@PA also showed stability for over one week or in PBS with 10% serum (FIG. 25i, FIG. 25d). However, liposome@PA precipitated after one-week storage when the loading rate increased to 20% (FIG. 25e). Overall, LPHNPs exhibited a notable advantage over the traditional liposome formulations.

### Optical and Photodynamic Properties.

We next investigated the optical properties for further understanding the co-assembly details of lipid and PQC molecules. As shown in FIG. 26a-FIG. 26b, similar to that of liposome@PA, the fluorescence of LPHNPs was quenched in the aggregation state (intact nanoparticle), while was released in the free state (dissociated nanoparticle). As previously shown, PA is a typical fluorophore of the aggregation-caused quenching (ACQ). The fluorescence quenching behavior of LPHNPs indicates that the strong hydrophobicity interaction and π-π stacking of PA are essential roles in the co-assembly. Moreover, the near-infrared (NIR) fluorescence imaging studies also supported similar findings, such as the inactivated fluorescence of aggregated photosensitizer and the discernible fluorescence of free ones (FIG. 26c). Subsequently, the photodynamic efficiency was evaluated by using the singlet oxygen sensor green (SOSG) as an ¹O₂ indicator. It was found that in the same medium, different formulations of PQC and PA produced an equal level of ¹O₂ production. This result not only indicates that the PQC and PA with the same photosensitization group have the equivalent photodynamic efficiency but also implies that the co-assembly with lipid did not hinder the ¹O₂ generating capacity of PQC (FIG. 26i). In addition, the free PQC or PA molecules (in SDS/PBS) produced significantly increased levels of ¹O₂ than their aggregated forms (in pure PBS), which is consistent with the fluorescence performance and our previous findings.

### Cellular Uptake and Cell Viability.

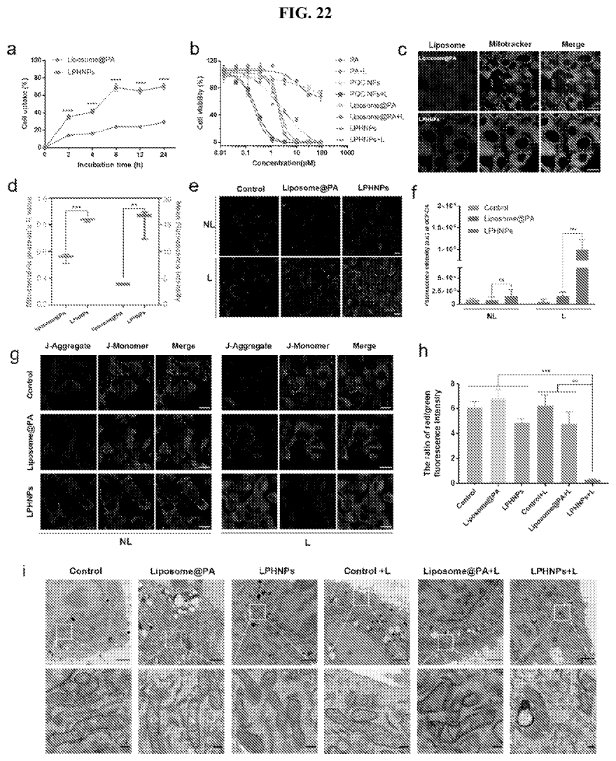

Cellular uptake of LPHNPs was assessed by using GL261, a murine glioma cell line. As shown in FIG. 22a, LPHNPs showed a rapid accumulation inside cells over time, and their cellular concentrations at predetermined time points are significantly higher than that of liposome@PA, respectively. This is caused because the neutral surface potential of LPHNPs is higher than that of the conventional liposomes. Cell viability assays were then carried out to ascertain the anticancer effects against GL261, and the results are presented in FIG. 22b. In the absence of light irradiation, liposome@PA and free PA exhibited a neglectable anti-GL261 effect (IC₅₀>90 μM), while LPHNPs and PQC NFs were more potent by showing their IC₅₀ at approximately 3 μM (FIG. 27c). Compared with the corresponding treatment group without light, each group involved in light exposure showed increased antiproliferative activities against GL261. Notably, among all groups, the light-activated LPHNPs and PQC NFs exhibited the most striking potency (IC₅₀=0.21 μM). Similar results were also validated in other two human glioma cell lines, U251 and U118 (FIG. 27a-FIG. 27b). It is worth mentioning that no matter with or without light irradiation, LPHNPs formed from the co-assembly of lipid and PQC are equivalent in antiproliferative efficiency to PQC NFs formed by the self-assembly of PQC, reflecting that the LPHNPs still preserve the similar properties of PQC NPs in the sub-localization and photosensitization inside cancer cells.

### Mitochondria Targeting and Mito-ROS Production.

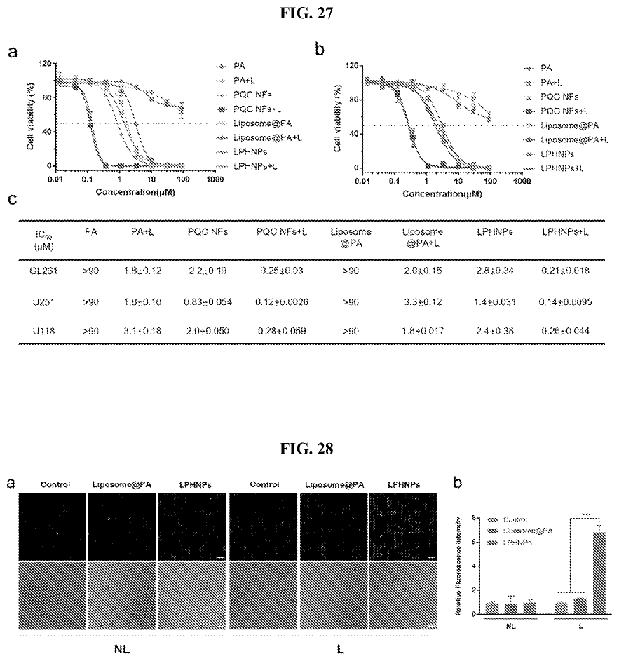

The mitochondria targeting localization is a key proponent to the compelling photodynamic anticancer effect of PQC NFs in cells. To clarify whether LPHNPs possess a comparable property of subcellular localization, the mitochondria colocalization study was conducted. GL261 cells were incubated with LPHNPs or liposome@PA, followed by staining with mitotracker green. As shown in FIG. 27c-FIG. 27d, the red fluorescence of LPHNPs consistently overlapped with the mitochondria signals, which contrasts sharply with the performance of liposome@PA and indicates that LPHNPs effectively deliver PQC molecules to mitochondria after uptake by cells. Since it has been verified that LPHNPs can target mitochondria of cancer cells, we then explored their cellular photosensitization effects at the cellular level. The mitochondrial ROS (mito-ROS) levels were measured by using MitoROS™ 580. Compared with liposome@PA, LPHNPs significantly enhanced the generation of ROS in mitochondria after irradiation (FIG. 28). Because the mitochondria are the main source of ROS, the enhanced mito-ROS level would lead to excessive cellular ROS. By using 2′,7′-dichlorofluorescein diacetate (DCF-DA), we measured the cellular ROS. As shown in FIG. 22e-FIG. 22f, groups upon light treatment showed enhanced ROS production compared with the groups without light treatment. Moreover, LPHNPs with light irradiation dramatically induced ROS generation, while liposome@PA NPs only exhibited a minor increase of ROS. This difference was attributed to the mitochondria targeting capacity and a higher cellular uptake of LPHNPs. Owing to the limited cellular uptake and lack of mitochondria-targeting ability, liposome@PA NPs only showed a small amount of ROS production.

### Mitochondria Dysfunction.

Mitochondria membrane potential (Δψₘ) is central to mitochondria functions, including driving ATP synthesis and keeping the balance of mitochondria metabolism. Decreased Δψₘ is a critical sign of mitochondria dysfunction. Thus, we detected the changes of Δψₘ by utilizing the JC-1 dye, which aggregates in mitochondria of normal Δψₘ and fluoresces red, and under low Δψₘ is dispersed emitting a green fluorescence. Compared to the untreated group, LPHNPs with irradiation caused a significant decline in the ratio of red to green fluorescence intensity, indicating that the mitochondria treated with LPHNPs plus light have a decreased Δψₘ (FIG. 22g-FIG. 22h). To further visualize the effect of LPHNPs on mitochondria microstructure, the cells that underwent different treatments were observed under TEM. As shown in FIG. 22i, mitochondria in the control and liposome@PA groups exhibited intact membrane structure, with the easily identifiable double membrane and arranged cristae. LPHNPs without light irradiation caused only a slight morphological change, reflecting that LPHNPs target mitochondria and function there. The mitochondria within in LPHNPs and irradiation treatment displayed disintegrated membranes and cristae, while those treated with liposome@PA and light remained the distinguishable membrane structure. These data demonstrated that LPHNPs led to severe mitochondria dysfunction by targeting mitochondria-targeted photodynamic effects.

### Fluorescence Imaging In Vivo.

To explore the systemic biocompatibility of LPHNPs, we assessed their safety in mice by monitoring body weight, hematology and pathological analysis. No significant body weight changes were observed after injection were observed (FIG. 29). H&E staining showed that the histological patterns of major organs of LPHNPs treated mice are similar to those of the PBS group, indicating minimal systemic toxicity of LPHNPs. In addition, there were no obvious changes in the hematologic indexes found among the two groups. These results suggested that LPHNPs are tolerated in mice with i.v. administration.

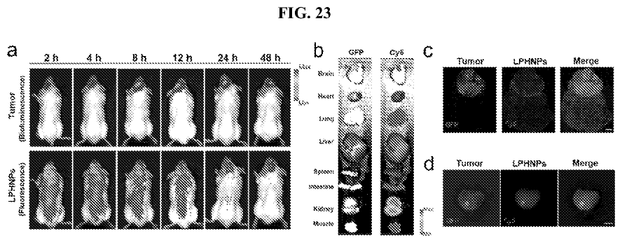

To examine the application of LPHNPs in tumor imaging, an orthotopic glioma model was established by implanting GL261 cells expressing GFP and luciferase into the right striatum area of C57 mouse. Fluorescence imaging in vivo was conducted by monitoring fluorescence of LPHNPs at different time intervals. FIG. 23a showed that LPHNPs circulated rapidly through the whole body and accumulated at the tumor region 2 h post-injection. Importantly, LPHNPs are mainly distributed in tumor site after 48 h injection, indicating its excellent tumor targeting capacity. The corresponding confocal imaging of cryo-sections exhibited strong overlapping between GL261 tumor (green) and LPHNPs (red) (FIG. 23c).

To further study the distribution of LPHNPs in vivo, the mice were sacrificed at 24 h post-injection to collect the major organs for ex vivo imaging. FIG. 23b, FIG. 23d showed that the fluorescent signals of LPHNPs highly overlapped with the GFP signals which indicated the tumor region, confirming the remarkable ability for brain tumor imaging of LPHNPs. The majority of collected organs showed low fluorescence signals, while the signals in kidney were relatively high. This is likely due to the renal clearance pattern for porphyrin derivative.

### Antitumor Efficacy In Vivo.

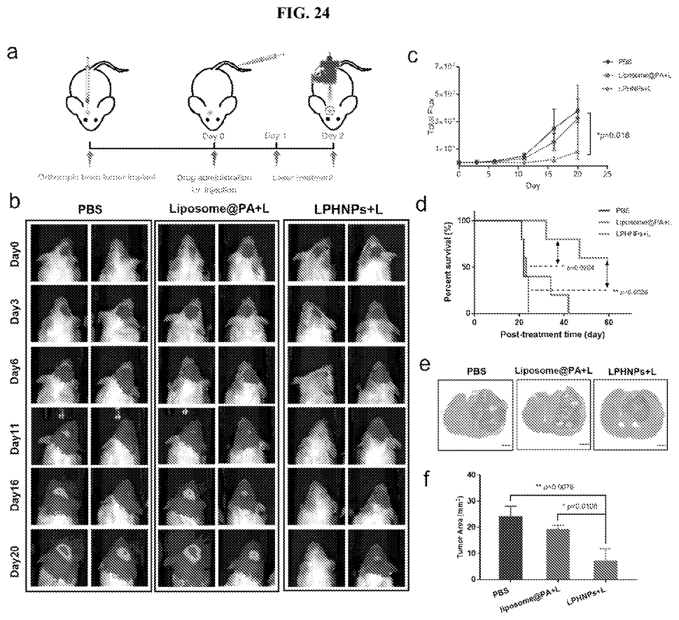

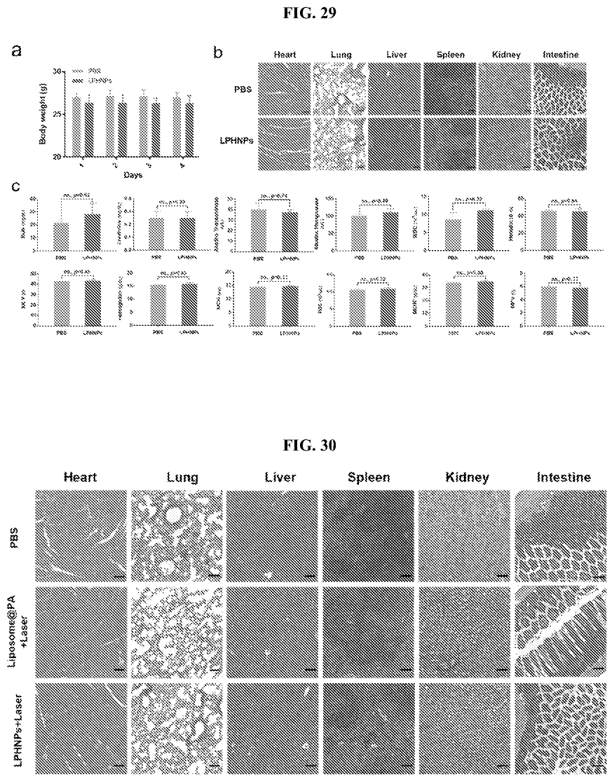

The therapeutic effect in vivo was evaluated by using the orthotopic GL261 model as well. Mice were randomly assigned into three groups (n=5) and treated as indicated, followed by two laser treatments (0.2 W/cm², 3 min) at 24 h and 48 h post-injection, respectively (FIG. 24a). As shown in FIG. 24b-FIG. 24d, liposome@PA with laser group negligibly inhibited tumor progression and did not prolong the overall survival time of mice compared to the PBS group showing progressively increased bioluminescence intensity. In contrast, LPHNPs with laser group significantly impeded tumor growth and extended overall survival outcome of animals (median survival, >60 days), as compared to PBS group (median survival, 22 days) and liposome@PA with laser group (median survival, 23 days). Importantly, three mice from the treatment group of LPHNPs plus laser lived longer than 60 days. The tumor tissues displayed apparent alterations among different treatment groups (FIG. 24e). Compared with other groups, the mice treated with LPHNPs and laser showed smaller tumor areas according to H&E staining (FIG. 24f). In addition, all groups didn't exhibit abnormalities in the histology of major organs, further indicating the safety of this hybrid nanoparticle in vivo (FIG. 30).

### Conclusion

In this work, we developed a mitochondria-targeting hybrid nanoparticle (LPHNPs) based on an amphiphilic photosensitizer and liposome as an efficient nanoplatform for glioma imaging and therapy. The amphiphilic photosensitizer, PQC, is loaded into the liposome with an enhanced loading rate, which overcomes the limited loading capacity limitation of conventional liposomes. The PEGylation and desirable nano-size of nanoparticle lead to its prolonged circulation time in body and drug accumulation in tumor sites due to the enhanced permeability and retention (EPR) effect. In addition, LPHNPs showed negligible systemic toxicity. Fluorescence imaging showed that LPHNPs were accumulated in tumor region at 2 h post-injection and retained for at least 48 h. Ex vivo imaging further confirmed the tumor targeting capacity of LPHNPs. Conventional photosensitizers exhibit limited ROS production due to a lack of mitochondria targeting capacity, which impeded therapeutic efficacy of PDT therapy. Thus, the mitochondria targeting PQC was utilized to endow LPHNPs with excellent mitochondrial targeting specificity. Under laser exposure, LPHNPs showed enhanced ROS and mito-ROS production in cells and resulted in mitochondria depolarization and structural damage. LPHNPs with irradiation group displayed ˜10 times lower IC50 value than that of liposome@PA NPs with irradiation. In the orthotopic glioma model, single dose of LPHNPs with laser treatment exhibited superior inhibition efficacy on tumor progression. More importantly, it dramatically extended the overall survival time of orthotopic glioma model compared with liposome@PA NPs and laser treated group. These results suggested that LPHNPs can serve as a promising theragnostic platform in glioma therapy.

### Example 5. LND-Mito Preparation

### Synthesis of LND-Mito

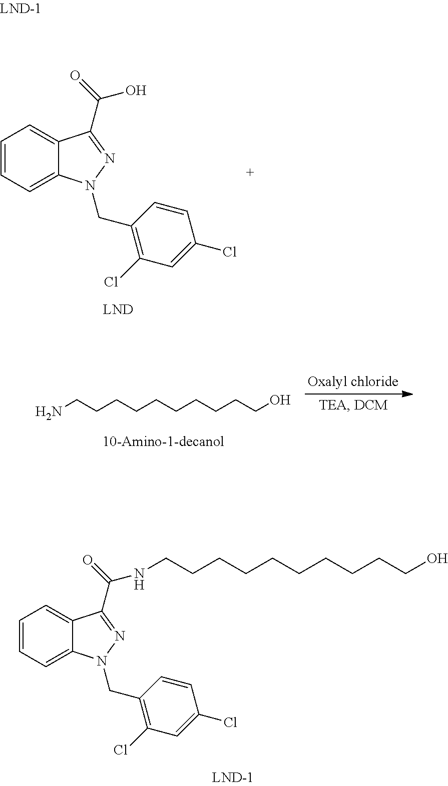 [SMILES: O=C(O)c1nn(Cc2ccc(Cl)cc2Cl)c2ccccc12 ; NCCCCCCCCCCO ; O=C(NCCCCCCCCCCO)c1nn(Cc2ccc(Cl)cc2Cl)c2ccccc12]

To a solution of 1-(2,4-dichlorobenzyl)-1H-indazole-3-carboxylic acid (ionidamine, 0.96 g, 3.0 mmol) in 30 ml dichloromethane, oxayl chloride (2 ml) and a catalytic amount of DMF (0.1 ml) were carefully added. The solution was heated under reflux for 6 h, and then was processed under reduced pressure for 30 min to remove the unreacted oxalyl chloride and solvent, which yields 1-(2,4-dichlorobenzyl)-1H-indazole-3-carbonyl chloride as a yellow solid. The solid, 10-amino-1-decanol (780 mg, 4.5 mmol) and trimethylamine (655 μl, 4.5 mmol) was dissolved in 20 ml of dichloromethane and was stirred for 12 h at room temperature. The reaction mixture was washed with water, brine, and dried over Na₂SO₄, followed by purification through silica column to afford LND-1, yield: 850 mg, 59.5%. HRMS-ESI [M+H]⁺ found 476.1884.

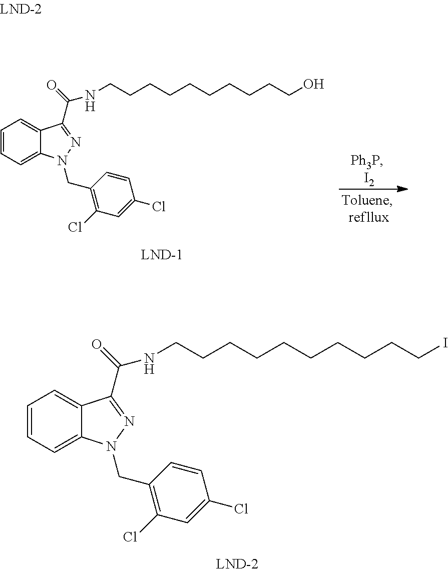 [SMILES: O=C(NCCCCCCCCCCO)c1nn(Cc2ccc(Cl)cc2Cl)c2ccccc12 ; O=C(NCCCCCCCCCCI)c1nn(Cc2ccc(Cl)cc2Cl)c2ccccc12]

LND-1 (953 mg, 2 mmol), triphenylphosphine (630 mg, 2.4 mmol) and imidazole (178 mg, 2.6 mmol) was dissolved in 30 mL of toluene. Iodine (609 mg, 2.4 mmol) was added portion wise at 0° C. The reaction mixture was reflux for 6 h, and absolute ethanol (3 mL) was added in two portions at around 10 min intervals. After evaporating solvent, the residue was purified by silica column to afford LND-2, Yield: 920 mg, 78.6%. HRMS-ESI [M+H]⁺ found 586.0914.

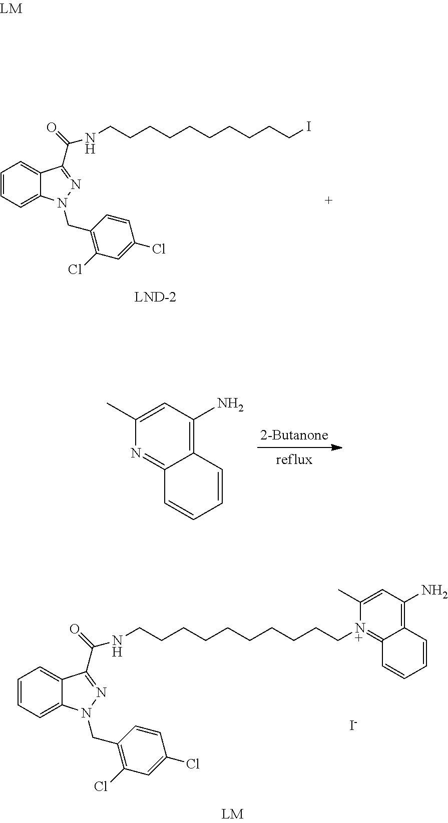 [SMILES: O=C(NCCCCCCCCCCI)c1nn(Cc2ccc(Cl)cc2Cl)c2ccccc12 ; Cc1cc(N)c2ccccc2n1 ; Cc1cc(N)c2ccccc2[n+]1CCCCCCCCCCNC(=O)c1nn(Cc2ccc(Cl)cc2Cl)c2ccccc12 ; [I-]]

The solution of LND-2 (1.17 g, 2 mmol) and 4-aminoquinaldine (253 mg, 1.6 mmol, 0.8 eq) in 2-butanone (30 mL) was refluxed at 95° C. for 4 d. The crude produce was purified via silica column to afford LM, Yield 450 mg, 37.8%. HNMR S 8.83 (2H, d, J=17.6 Hz), 8.45 (1H, d, J=8.0 Hz), 8.32 (1H, t, J=5.6 Hz), 8.22 (1H, d, J=8.8 Hz), 8.15 (1H, d, J=7.2 Hz), 8.03 (1H, td, J₁=7.2 Hz, J₂=0.8 Hz), 7.75 (3H, m), 7.48 (1H, td, J₁=8.0 Hz, J₂=0.8 Hz) 7.36 (1H, dd, J₁=8.0 Hz, J₂=2.4 Hz), 7.30 (1H, t, J=8.0 Hz), 6.77 (1H, d, J=8.0 Hz), 6.72 (1H, s), 5.82 (2H, s), 4.45 (2H, t, J=8.0 Hz), 3.28 (2H, m), 2.73 (3H, s), 1.73 (2H, m), 1.54 (2H, m), 1.45 (2H, m), 1.33 (10H, m). HRMS-ESI [M+H]⁺ 616.2604

### Example 6. LND-Mito Biological Assays

### In-Vitro Antitumor Activity

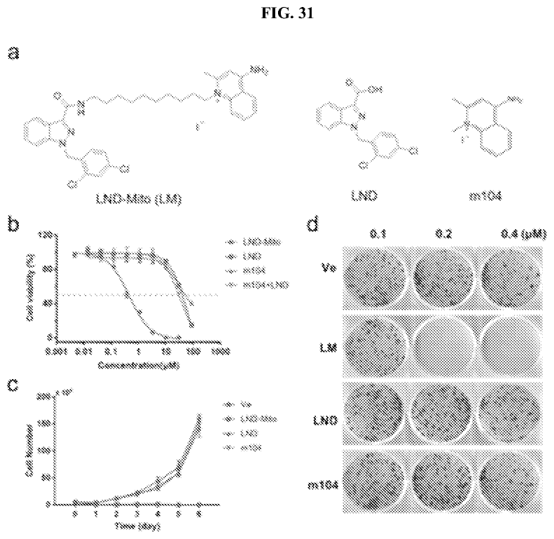

Cell viability assay. Pancreatic cancer stem cells (CSC, 5×10³ cells per well) was plated in 96-well plates, incubated overnight, and then were treated with different concentrations of agents as indicated. After 72 h treatment, cell viability was quantified using the CellTiter-Glo assay Cell viability curves and the chemical structures of LM, LND, and m104 are shown in FIG. 31A and FIG. 31B. LND, an antiglycolytic drug, showed limited efficacy against CSC cells. LM shows significantly enhanced potent than LND, m104, mixture of LND and m104. (IC₅₀ are 0.46±0.05 μM, 30±2.2 μM, 31±7.5 μM, 36±6.8 μM respectively).

Cell growth. Pancreatic cancer stem cells were plated in 6-well plates (50,000 cell per well) and treated as indicated and were counted manually every 24 h. The cell growth is plotted over time in FIG. 31C. The results of the cell growth assay demonstrated the inhibitory effects of LM on tumor cells.

Colony formation. Colony formation assay was performed on 6-well plates with a starting density of 2000 cells per well. After incubated as indicated for 14 days, cells were washed with PBS and stained with the solution of crystal violet for 20 min. The clonogenic assay of CSC cells treated as indicated in FIG. 31D.

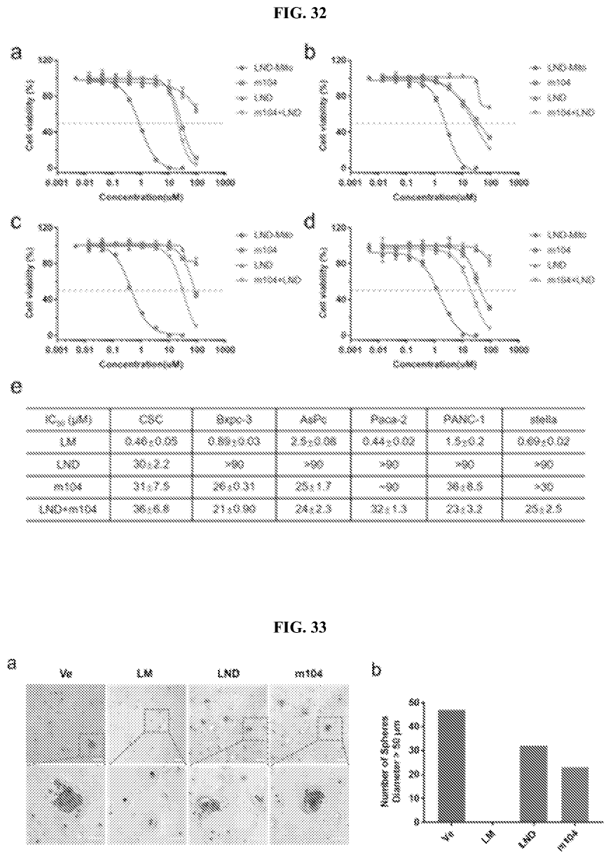

Cell viability. Bxpc-3 and AsPc cells (CSC, 5×10³ cells per well) was plated in 96-well plates, incubated overnight, and then were treated with different concentrations of agents as indicated. After 72 h treatment, cell viability was quantified using the CellTiter-Glo assay. LM shows more potent than LND, m104, mixture of LND and m104 on other two types of pancreatic tumor cells. The cell viability curves are shown in FIG. 32A and FIG. 32B. IC₅₀ values of cytotoxicity were calculated by GraphPad 8 and are shown in FIG. 32C.

Sphere formation. Pancreatic cancer stem cells (3000 cells per well) were mixed with matrigel and medium, and then placed in 24-well plates with pre-embedded matrigel and medium. After 24 h incubation, CSC cells were then treated with different agents as indicated and cultured for 10 days. The cells were imaged as shown in FIG. 33A and the sphere diameters were calculated by ImageJ and are plotted in FIG. 33B. The results indicated that LM blocked tumorsphere formation in CSCs while LND and m104 have limited inhibition.

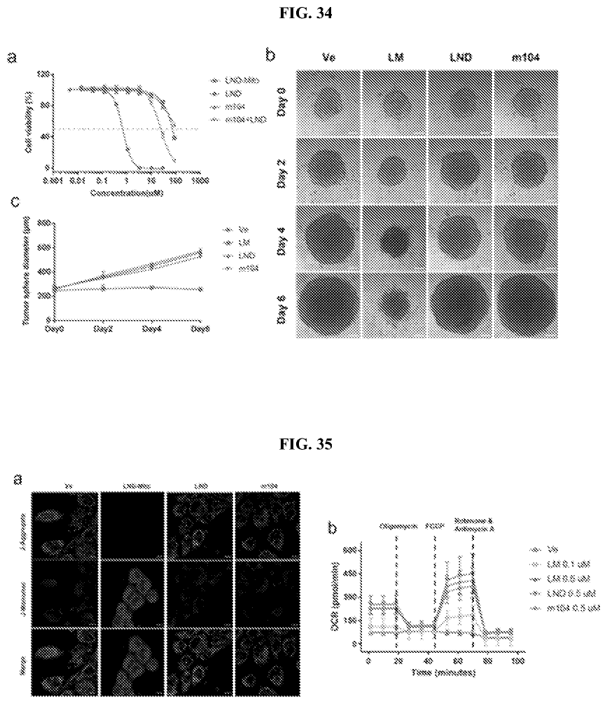

Sphere growth. Stella cells (5×10³ cells per well) were plated in 96-well plates, incubated overnight, and then were treated with different concentrations of agents as indicated. After 72 h treatment, cell viability was quantified using the CellTiter-Glo assay. The cell viability curves are shown in FIG. 34A. CSC cells (800 cells per well) and stella cells (400 cells per well) were plated together in 96-well low-attachment plates and incubated overnight. CSC spheres then were treated with different agents as indicated. Spheres growth was monitored by CLSM (every two days). The images of the cells are shown in FIG. 34B. The sphere diameters were calculated by ImageJ for the CSC spheres (with Stella cell) after treatment (LM, LND, and m104; 5 μM) as shown in FIG. 34C. The results indicated that LM exhibit similar inhibitory effects on Stella cell. LM inhibits CSC tumorsphere (with Stella cell) growth, while LND and m104 have no effect.

Mitochondria membrane potential. JC-1 dye was used as an indicator of mitochondrial membrane potential. Briefly, Cells (2×10⁴ cells per well) were treated as indicated for 24 h, washed and cultured with fresh medium. Then 0.5 μg/mL JC-1 was added for another 30 min incubation. Images were captured by CLSM. The analysis is shown in FIG. 35A. The results indicated that LM caused a significant decline in red fluorescence intensity and an increase of green fluorescence, indicating that the mitochondria treated with LM have a decreased Δψₘ, which is a critical sign of mitochondria dysfunction.

Seahorse assay. CSC cells (2×10⁴ cells per well) were treated as indicated for 24 h. The Oxygen Consumption Rate (OCR) were measured by the Agilent Seahorse XF analyzer. The metabolic flux analysis of CSC treated as indicated are shown in FIG. 35B. The Mito stress test reveals a decrease in basal mitochondrial respiration and spare respiration capacity in CSC cells after treatment with LM. Compared to vehicle group, LND and m104 have no significant impact on OCRs.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula (I): 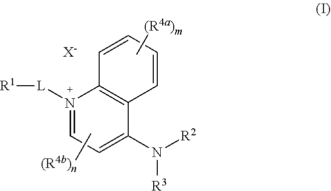
wherein:
R¹ is a hydrophobic drug or photosensitizer;
L is C₂₋₂₀ alkylene, C₂₋₂₀ alkenylene, C₂₋₂₀ alkynylene, or polyethyleneglycol polymer;
R² is H, C₁₋₆ alkyl, 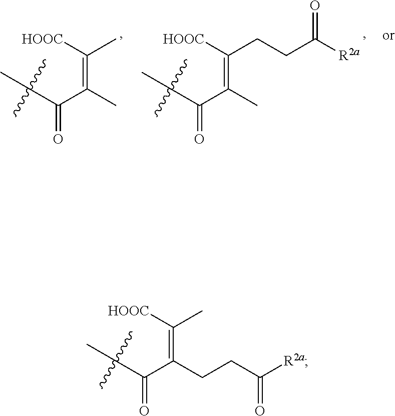
R^{2a} is —OH, —NH-polyethyleneglycol, polyethyleneglycol or a peptide;
R³ is H or C₁₋₆ alkyl;
each R^{4a} and R^{4b} is independently H, C₁₋₆ alkyl, C₂₋₆ alkenyl, C₂₋₆ alkynyl, halogen, or C₁₋₆ haloalkyl;
subscript m is from 1 to 4;
subscript n is from 1 to 2; and
X is Cl, Br or I;
wherein when R¹ is cyclosporin, R² is Me, and R³ is Me, then L is C₂₋₂₀ alkylene, C₁₀₋₂₀ alkenylene, C₂₋₂₀ alkynylene, or polyethyleneglycol polymer.

2. The compound of claim 1, wherein R¹ is the hydrophobic drug is indazole-3-carboxylic acid, lonidamine, tolnidamine, steroids, triterpenoids, botulin, β-lapachone, vitamin E, α-tocopheryl, α-tocopheryl succinate, or derivatives thereof.

3. The compound of claim 1, wherein R¹ is lonidamine.

4. The compound of claim 1, wherein R¹ is the photosensitizer.

5. The compound of claim 4, wherein the photosensitizer is a porphyrin.

6. The compound of claim 1, wherein L is C₂₋₂₀ alkylene.

7. The compound of claim 1, wherein L is C₁₀ alkylene.

8. The compound of claim 1, wherein R² and R³ are each hydrogen.

9. The compound of claim 1, wherein
each R^{4a} is H; and
each R^{4b} is independently H or C₁₋₆ alkyl.

10. The compound of claim 1, wherein
each R^{4a} is H; and
each R^{4b} is independently H or methyl.

11. The compound of claim 1, wherein X is I⁻.

12. The compound of claim 1, wherein the compound is:

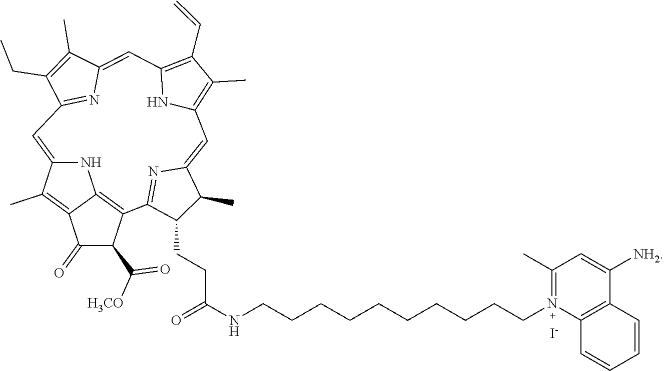

13. The compound of claim 1, wherein the compound is:

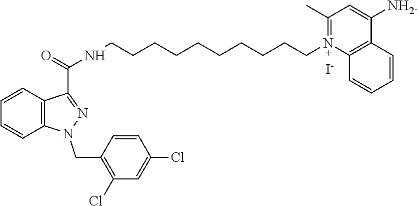

14. A nanofiber comprising a plurality of conjugates of Formula (I): 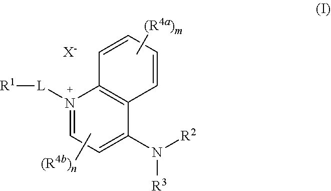
wherein:
R¹ is a hydrophobic drug or photosensitizer;
L is C₂₋₂₀ alkylene, C₂₋₂₀ alkenylene, C₂₋₂₀ alkynylene, or polyethyleneglycol polymer;
R² is H, C₁₋₆ alkyl, 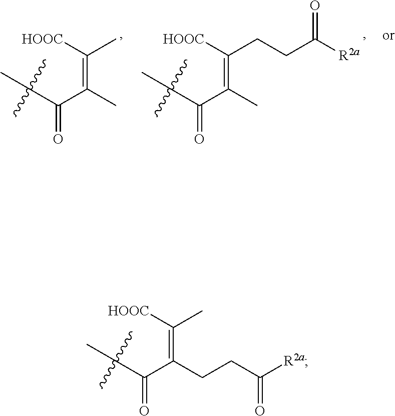
R^{2a} is —OH, —NH-polyethyleneglycol, polyethyleneglycol or a peptide;
R³ is H or C₁₋₆ alkyl;
each R^{4a} and R^{4b} is independently H, C₁₋₆ alkyl, C₂₋₆ alkenyl, C₂₋₆ alkynyl, halogen, or C₁₋₆ haloalkyl;
subscript m is from 1 to 4;
subscript n is from 1 to 2; and
X is Cl, Br or I.

15. The nanofiber of claim 14, wherein each conjugate of Formula (I) is the compound:

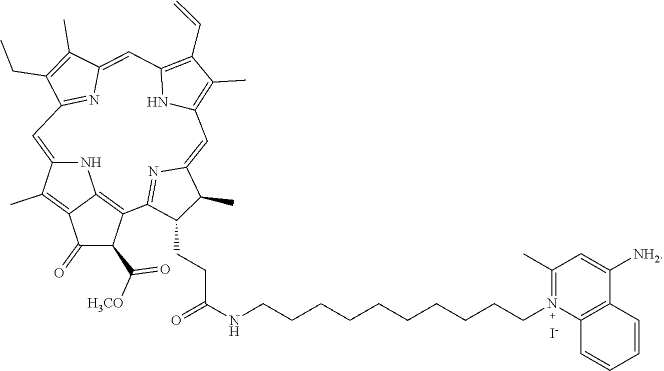

16. The nanofiber of claim 14, wherein each conjugate of Formula (I) is the compound:

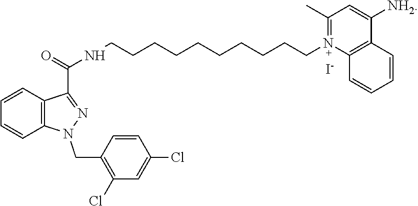

17. A nanoparticle comprising a plurality of conjugates of Formula (I): 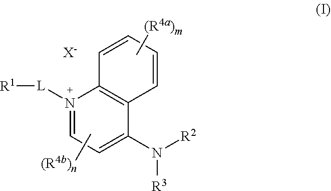
wherein:
R¹ is a hydrophobic drug or photosensitizer;
L is C₂₋₂₀ alkylene, C₂₋₂₀ alkenylene, C₂₋₂₀ alkynylene, or polyethyleneglycol polymer;
R² is H, C₁₋₆ alkyl, 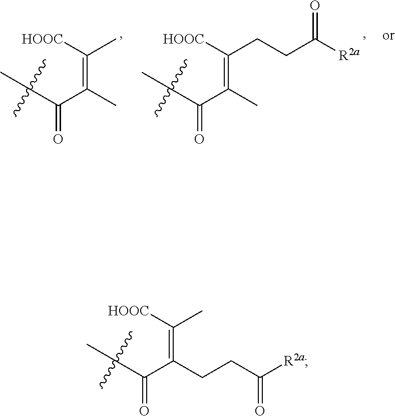
R^{2a} is —OH, —NH-polyethyleneglycol, polyethyleneglycol or a peptide;
R³ is H or C₁₋₆ alkyl;
each R^{4a} and R^{4b} is independently H, C₁₋₆ alkyl, C₂₋₆ alkenyl, C₂₋₆ alkynyl, halogen, or C₁₋₆ haloalkyl;
subscript m is from 1 to 4;
subscript n is from 1 to 2; and
X is Cl, Br or I.

18. The nanoparticle of claim 17, wherein each conjugate of Formula (I) is the compound:

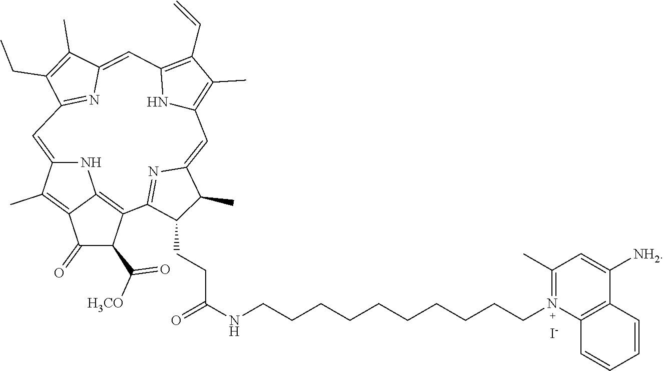

19. The nanoparticle of claim 17, wherein each conjugate of Formula (I) is the compound:

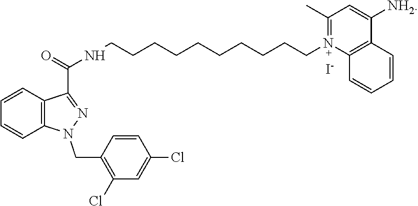

20. The nanoparticle of claim 17, wherein the nanoparticle comprises 1-alpha-phosphatidylcholine, cholesterol, and mPEG-DSPE.

21. A method of treating a disease, the method comprising administering a therapeutically effective amount of a nanofiber of claim 14 to a subject in need thereof.

22. (canceled)

23. (canceled)

24. A method of treating a disease via photodynamic therapy, the method comprising administering a therapeutically effective amount of a nanofiber of claim 14, wherein R¹ is a photosensitizer, to a subject in need thereof.

25.-30. (canceled)
